(19) United States
(12) Patent Application Publication
(10) Pub. No.: US 2016/0235686 A1
(43) Pub. Date: Aug. 18, 2016

(54) PROCESSES FOR PREPARING A SOLID POLYMER MATRIX CONTAINING A CORE MATERIAL BY PRESSURE CYCLING OF SUPERCRITICAL FLUID

(71) Applicant: CRITICAL PHARMACEUTICALS LIMITED, Nottingham (GB)

(72) Inventors: Andrew NAYLOR, Nottingham (GB); Mark Andrew WHITAKER, Nottingham (GB); Nicholas Jon ARROWSMITH, Nottingham (GB); Gregoire Charles Joseph SCHWACH, Copenhagen (DK)

(73) Assignee: CRITICAL PHARMACEUTICALS LIMITED, Nottingham (GB)

(21) Appl. No.: 15/027,978

(22) PCT Filed: Oct. 7, 2014
(86) PCT No.: PCT/GB2014/053024

§ 371 Date: Apr. 7, 2016

(30) Foreign Application Priority Data
Oct. 8, 2013 (GB) 1317756.3

(51) Int. Cl. A61K 9/50; A61K 38/27

(52) U.S. Cl. CPC A61K 9/5089; A61K 9/5031; A61K 38/27

(57) ABSTRACT

There is provided a supercritical fluid-based process for preparing a solid polymer matrix containing a core material, wherein the process includes the step of mixing the polymer, core material and supercritical fluid in a mixing vessel, followed at least one cycle of, without recovering the solid polymer matrix, (i) converting the supercritical fluid in the mixing vessel to a sub-critical state, and then (ii) returning the fluid to the supercritical state, provided that the core material does not comprise any of gonadotropin releasing hormone (GnRH), a GnRH agonist and a GnRH antagonist.

The present invention relates to a process for preparing a solid polymer matrix containing a core material. The invention also relates to solid polymer matrices that are obtainable by such a process. Such solid matrices can be used to provide, for example, sustained and/or delayed release of core material from a polymer.

The listing or discussion of an apparently prior-published document in this specification should not necessarily be taken as an acknowledgement that the document is part of the state of the art or is common general knowledge.

Methods for the production of compositions comprising a core material and a polymer using a supercritical fluid have been reported in the past.

U.S. Pat. No. 5,340,614, WO 91/09079 and U.S. Pat. No. 4,598,006 describe methods for providing bioactive material in a biodegradable polymer using supercritical fluids (SCF) to confer porosity during processing of the polymer.

U.S. Pat. No. 5,340,614 describes a method comprising dissolution of additive in a carrier solvent (liquid e.g. water or ethanol). A supercritical fluid (SCF) is then used to allow penetration of the carrier liquid/additive solution into the polymer.

WO 91/09079 describes the use of SCF to introduce porosity into biodegradable polymers. If a bioactive material is present, a carrier solvent is required to dissolve the bioactive and to impregnate.

U.S. Pat. No. 4,598,006 describes a method for impregnating a thermoplastic polymer with an impregnation material in a volatile swelling agent at or near supercritical conditions, swelling the polymer and reducing the conditions so that the swelling agent diffuses out.

WO 98/15348, WO 98/51347 and WO 2003/078508 describe methods for the encapsulation of materials within a polymer matrix, without the use of solvents or high temperatures. A supercritical fluid is used to depress the melting or glass transition temperature of the polymer so that the material can be encapsulated within the polymer at low temperatures and in the absence of organic or aqueous solvents.

WO 03/013478 also describes a method of encapsulating an active substance in an interpolymer complex using supercritical fluids. Methods are described involving the dissolution of an interpolymer complex, or components thereof, in a supercritical fluid, or the dissolution of a supercritical fluid in an interpolymer complex. In both these systems an active substance is then encapsulated.

WO 2010/004287 describes how certain processing aids can be used to provide improved methods of encapsulating certain materials into polymers using supercritical fluids.

However, there remains a need for supercritical fluid-based processes that are able to impart improved properties to the polymeric composite resulting from encapsulation of a material in the polymer. For example, there remains a need for such processes that are able to provide polymeric composites having enhanced (e.g. more sustained) release profiles of the encapsulated material.

In a first aspect, the present invention relates to a process for preparing a solid polymer matrix containing a core material, said process comprising the steps of:
(a) providing a solid polymer, a core material and a fluid that is capable of existing in the supercritical state;
(b) in a mixing vessel, mixing the polymer, core material and fluid at
   a temperature at or above T_{c} and
   a pressure at or above P_{c},
   such that the fluid is in the supercritical state, wherein T_{c} and P_{c} are the critical temperature and the critical pressure, respectively, for the fluid;
(c) without recovering the solid polymer matrix,
   (i) converting the fluid in the vessel to a sub-critical state by reducing the pressure in said vessel to below P_{c} and/or reducing the temperature in said vessel to below T_{c}, and then
   (ii) returning the fluid in the vessel to the supercritical state by increasing the pressure and/or the temperature in the vessel;
(d) optionally repeating step (c) one or more times; and
(e) releasing the pressure in the vessel and recovering solid polymer matrix containing the core material,
   provided that the core material does not comprise any of gonadotropin releasing hormone (GnRH), a GnRH agonist and a GnRH antagonist,
   which process may hereinafter be referred to as “the process of the invention”.

In particular embodiments of the first aspect of the invention, the process is carried out in the presence of a processing aid. In such embodiments, the process comprises the steps of:
(a1) providing a solid polymer, a core material, a processing aid and a fluid that is capable of existing in the supercritical state;
(b1) in a mixing vessel, mixing the polymer, core material, processing aid and fluid at
   a temperature at or above T_{c} and
   a pressure at or above P_{c},
   such that the fluid is in the supercritical state, wherein T_{c} and P_{c} are the critical temperature and the critical pressure, respectively, for the fluid;
(c1) without recovering the solid polymer matrix,
   (i) converting the fluid in the vessel to a sub-critical state by reducing the pressure in said vessel to below P_{c} and/or reducing the temperature in said vessel to below T_{c}, and then
   (ii) returning the fluid in the vessel to the supercritical state by increasing the pressure and/or the temperature in the vessel;
(d1) optionally repeating step (c) one or more times; and
(e1) releasing the pressure in the vessel and recovering solid polymer matrix containing the core material,
   provided that the core material does not comprise any of gonadotropin releasing hormone (GnRH), a GnRH agonist and a GnRH antagonist.

Gonadotropin releasing hormone is also known as luteinising hormone releasing hormone (LHRH). Thus, the processes of the invention specifically exclude the use of LHRH, LHRH agonists and LHRH antagonists (as these are identical to GnRH, GnRH agonists and GnRH antagonists, respectively).

The structure of GnRH is well known to those skilled in the art and is as follows.
pyroGlu-His-Trp-Ser-Tyr-Gly-Leu-Arg-Pro-Gly-NH₂

When used herein, the term “GnRH agonist” refers to molecules that bind to the GnRH receptor and elicit release of Follicle-stimulating hormone (FSH) and/or Luteinizing hormone (LH). In this respect, the term “GnRH agonist” specifically includes references to buserelin, deslorelin, goserelin, histrelin, leuprolide, nafarelin and triptorelin.

When used herein, the term “GnRH antagonist” refers to molecules that bind to the GnRH receptor but that do not elicit release of FSH or LH. In this respect, the term “GnRH antagonist” specifically includes references to abarelix, cetrorelix, degarelix, ganirelix and teverelix.

Whether or not a molecule binds to the GnRH receptor and/or elicits release of FSH and/or LH upon binding to that receptor can be determined by methods that are well known to those skilled in the art. For example, binding to the GnRH receptor can be determined by use of ELISA (an enzyme-linked immuno sorbent assay). Further, release of FSH and/or LH can be determined in vivo, for example, by administration of the molecule to a subject (e.g. a mammal such as a rat, particularly an immature female rat), followed by quantification of the FSH and/or LH release by radioimmunoassay or other methods known to those skilled in the art (see, for example, Endocrinology, 144(4), 1380-92 (2003) and Neuro Endocrinol. Lett., 32(6), 769-73 (2011)).

In a second aspect, the invention relates to a solid polymer matrix containing a core material that is obtainable by (or is obtained by) a process according to the first aspect of the invention, provided that the core material does not comprise any of gonadotropin releasing hormone (GnRH), a GnRH agonist and a GnRH antagonist.

In a third aspect of the invention, there is provided a process for preparing a pharmaceutical composition comprising a solid polymer matrix that contains a core material, wherein the core material is a biologically active material, provided that the core material does not comprise any of gonadotropin releasing hormone (GnRH), a GnRH agonist and a GnRH antagonist,

said process comprising a process according to the first aspect of the invention, followed by a step of formulating the solid polymer matrix for pharmaceutical use.

The skilled person will appreciate that the solid polymer matrix referred to in the second aspect will be suitable for use as a pharmaceutical and may, therefore, be referred to as a pharmaceutical composition comprising the solid polymer matrix.

Methods of formulating polymer-encapsulated products for pharmaceutical use are well known to those skilled in the art. Particular pharmaceutical compositions that may be mentioned include those for subcutaneous (SC or s.c.) injection or, more particularly, intramuscular (IM or i.m.) injection, which compositions may be provided in the form of a suspension (i.e. a suspension of the solid polymer matrix in a pharmaceutically acceptable carrier, such as an aqueous carrier or an oily vehicle). Further, biologically active materials are as defined hereinafter.

### The (Solid) Polymer

When used herein, the term “solid polymer” refers to a polymer that is solid at ambient temperature (e.g. 298 K) and pressure (e.g. atmospheric pressure, such as 1 atmosphere). By “solid” it is meant that the polymer exhibits zero flow. Examples of solid polymers include amorphous polymers (at below their glass transition temperature, T_{g}), crystalline polymers (at below their melting temperature, Tₘ) or mixed crystalline/amorphous polymers (at below their T_{g} and Tₘ).

As discussed in more detail below, the invention encompasses the use of mixtures of two or more different polymers. For the purposes of obtaining a solid polymer matrix, it is sufficient that at least one (but not necessarily all) of the component polymers are solid at ambient temperature and pressure.

The polymer used in the present invention may be a single polymer or a mixture of two or more polymers. For example, two, three, four or more polymers may be used. Herein the reference to “the polymer” or “a polymer” is intended to encompass the plural unless the context indicates otherwise.

Any solid polymer that is capable of being swelled and/or plasticized by a supercritical fluid may be used in the process of the invention. Thus, the skilled person will understand that particular polymers that may be used in the process of the invention include solid polymers that are capable of being platicized by a supercritical fluid (such as supercritical carbon dioxide).

As used herein, references to a polymer being placitized may also include references to the polymer being liquefied. As used herein, the term liquefied will be understood to refer to a substance taking on the consistency of a liquid, which may be defined as being a single continuous mass that is capable of being stirred.

The skilled person will understand that references to the solid polymer being capable of being swelled and/or plasticized by a supercritical fluid in the process of the invention will include references to solid polymers that when used in the process of the invention can be shown to be (or have been) swelled and/or plasticized (e.g. plasticized). For example, a polymer can be shown to be plasticized if during the process of the invention that polymer takes on the consistency of a liquid (e.g. a viscous liquid), as will be readily recognisable by a person skilled in the art (e.g. due to the ability to stir the polymer as a single continuous mass).

Solid polymers that may be mentioned include:
synthetic biodegradable polymers, such as those disclosed in “Polymeric Biomaterials” ed. Severian Dumitriu, ISBN 0-8247-8969-5, Publ. Marcel Dekker, New York, USA, 1994 (incorporated herein by reference);
synthetic non-biodegradable polymers; and
natural polymers.

The polymer may be selected from homopolymers, block and random copolymers and polymeric blends, any of which may be straight chain, (hyper) branched or cross-linked.

Non-limiting examples of polymers which may be used in the process of the invention include those listed below.

Synthetic biodegradable polymers that may be mentioned include:
polyesters, including
   polyhydroxyacids (PHAs), such as poly(lactic acid) (PLA), poly(glycolic acid) (PGA), copolymers of lactic and glycolic acid (PLGA), copolymers of lactic and glycolic acid with poly(ethyleneglycol), poly(e-caprolactone) (PCL) and poly(3-hydroxybutyrate) (PHB),
   poly (ether esters), such as poly(p-dioxanone),
   polymers of diacids and diols, such as poly(propylene fumarate), poly(butylene terephthalate) and poly(alkylene oxalates),
   poly(ether ester) multiblock copolymers, such as polymers based upon poly(ethylene glycol) and poly(butylene terephthalate);
poly(ortho esters), including polyol/diketene acetals addition polymers as described by Heller in ACS Symposium Series 567, 292-305, 1994 (incorporated herein by reference);
polyanhydrides, such as poly(sebacic anhydride) (PSA), poly(carboxybiscarboxy phenoxyphenoxyhexane) (PCPP), poly[bis(p-carboxyphenoxy) methane] (PCPM), copolymers of SA, CPP and CPM, as described by Tamada and Langer in Journal of Biomaterials Science-Polymer Edition, 3, 315-353,1992 and by Domb in Chapter 8 of the Handbook of Biodegradable Polymers, ed. Domb A. J. and Wiseman R. M., Harwood Academic Publishers (both of which are incorporated herein by reference);
poly(amino acids);
poly(pseudo amino acids), such as those described by James and Kohn in pages 389-403 of Controlled Drug Delivery Challenges and Strategies, American Chemical Society, Washington DC (incorporated herein by reference);
polyphosphazenes, such as derivatives of poly[(dichloro) phosphazene], poly[(organo) phosphazenes], polymers described by Schacht in Biotechnology and Bioengineering, 52, 102-108, 1996 (incorporated herein by reference); and
azo polymers, such as those described by Lloyd in International Journal of Pharmaceutics, 106, 255-260, 1994 (incorporated herein by reference).

Synthetic non-biodegradable polymers that may be mentioned include:
vinyl polymers, such as polyethylene, polyvinylpyrrolidone, poly(ethylene-co-vinyl acetate), polypropylene, poly(vinyl chloride), poly(vinyl acetate), poly(vinyl alcohol), copolymers of vinyl alcohol and vinyl acetate, poly(acrylic acid) poly(methacrylic acid), polyacrylamides, polymethacrylamides, polyacrylates, polyacrylonitrile and polystyrene and its derivatives;
polyethers such as poly(ethylene glycol) (PEG), poly(propylene glycol) and copolymers (e.g. block copolymers) of ethylene glycol and propylene glycol;
silicone polymers such as poly(dimethyl siloxane);
polyurethanes; and
polycarbonates.

Natural polymers that may be mentioned, including, include:
carbohydrates, such as starch, cellulose, dextran, alginates (e.g. alginic acid and salts thereof) and hyaluronates (e.g. hyaluronic acid and salts thereof);
modified carbohydrates, such as chitin (a polymer of N-acetyl glucosamine);
polypeptides;
proteins, such as collagen; and
semi-synthetic polymers derived from such natural polymers, including
   cellulose derivatives, such as ethylcellulose, methylcellulose, ethylhydroxy-ethylcellulose and sodium carboxymethylcellulose,
   starch derivatives, such as hydroxyethyl starch;
   chitin derivatives, such as chitosan,
   protein derivatives, such as gelatin.

In embodiments of the invention that may be mentioned, the polymer comprises one or more of the following:
non-biodegradable polymers such as ester urethanes or epoxy, bis-maleimides, methacrylates such as methyl or glycidyl methacrylate, tri-methylene carbonate, di-methylene tri-methylene carbonate;
biodegradable synthetic polymers such as PGA, PLA, PLGA, poly(p-dioxanone), poly(alkylene oxalates), modified polyesters such as poly(ether ester) multiblock copolymers such as those based on poly(ethylene glycol) and poly(butylene terephthalate), and PCL.

In more particular embodiments of the invention that may be mentioned, the polymer comprises one or more of PCL, PHB, poly(ether ester) multiblock copolymers, PLGA and PLA (e.g. the polymer comprises PLGA, PLA, or a combination of PLA and PLGA).

In certain embodiments of the invention, the polymer is one of the polymers set out above. For example, the polymer may be a PHA, such as a PLA, a PGA or, particularly, a PLGA.

In certain other embodiments of the invention, the polymer is a mixture of two or more of the polymers set out above. For the avoidance of doubt, the two or more polymers may be from the same class (e.g. polyesters) or from two different classes (e.g. a polyester and a polyanhydride). In this respect, the polymer may, for example, be a mixture of two or more of:

(i) a first polyester (e.g. PLGA);
(ii) a second polyester (e.g. PLA or PGA); and
(iii) a polyether (e.g. PEG or, particularly, a random or, particularly, a block copolymer of ethylene glycol and propylene glycol, such as a triblock copolymer comprising two blocks of polyethylene glycol connected by a block of polypropylene glycol (e.g. a poloxamer (Synperonic, Pluronic or Kolliphor) such as PL407, otherwise known as Kolliphor P407)).

PLGA is poly(lactic-co-glycolic acid). The amount of lactic acid and glycolic acid comonomers present in the PLGA which may be used may vary over a wide range. Thus, in certain embodiments of the invention in which the polymer is or comprises PLGA, the PLGA has a molar ratio of lactic acid:glycolic acid of from about 90:10 to about 10:90, such as from about 75:25 to about 25:75, for example about 50:50.

The molecular weight of a polymer is related to its inherent viscosity. In certain embodiments of the invention that may be mentioned, the inherent viscosity of the polymers that may be used in the process of the invention (e.g. PLGA and PLA) may be from about 0.1 to about 1.5 dL/g. For example, the inherent viscosity (e.g. of a PLGA and/or a PLA component of the polymer) may be from about 0.11 to about 1.00 dL/g or about 0.12 to about 0.50 dL/g, for example from about 0.15 to about 0.30 dL/g or about 0.16 to about 0.24 dL/g. In particular embodiments of the invention that may be mentioned, the inherent viscosity (e.g. of a PLGA and/or a PLA component of the polymer) is from about 0.05 to about 0.15 dL/g (such as about 0.10 dL/g).

In more particular embodiments of the invention, the polymer comprises both PLGA and PLA, (and, optionally, a poloxomer such as PL407). In such embodiments, the ratio (by weight) of PLGA:PLA is typically from about 95:5 to about 5:95, such as from about 90:10 to about 40:60 (e.g. from about 85:15 to about 50:50, such as from about 75:25 to about 60:40). Further, when a poloxomer is present, the weight of poloxomer is typically from about 5 to about 25% of the combined weight of PLGA and PLA (e.g. from about 8 to about 15% or, particularly, from about 10 to about 12% of the combined weight of PLGA and PLA).

Preferably, the compositions produced by the process of the invention are “true blends” as opposed to phase-separated blends. By “true blends” we include the meaning that the compositions are well blended in a single, solvent free step. Differential scanning calorimetry (DSC) can be used to determine whether a true blend or a phase separated blend is obtained. This is explained in more detail below.

The or each solid polymer present in the compositions produced by the process of the invention will have a glass transition temperature (T_{g}), a melting temperature (Tₘ) or both a T_{g} and Tₘ.

A true-blended composition displays a single T_{g} (as measured by DSC) for the blend of solid polymers. In contrast, in a phase-separated blend, the T_{g} of the or each solid polymer component will tend to remain distinct from the or each T_{g} of the other solid polymer components.

In specific embodiments of the invention, the polymer comprises or consists of polymeric material(s) that is(are) inert to the core material to be incorporated into the polymer matrix.

The polymer can be present in any amount that enables formation of a solid polymer matrix containing the core material. In this respect, the polymer may represent, for example, from about 5 to about 99.9% by weight of the product of the process of the invention, namely the solid polymer matrix containing the core material (e.g. the weight of polymer is from about 5 to about 99.9% of the combined weight of the polymer and the core material). In certain embodiments of the invention, the weight of polymer is from about 25 to about 97, 98 or 99%, such as from about 45 to about 93% (e.g. from about 60 to about 85%) of the combined weight of the polymer and the core material.

### The Core Material

The core material can be any material capable of inclusion within a solid polymer matrix (e.g. for the purpose of achieving delayed and/or sustained release of that material from the polymer matrix).

The core material may, for example, be:
(a) in any physical form (e.g. a form selected from solid, semi-solid (e.g. thixotrope or gel), semi-fluid or fluid (e.g. paste of liquid), such as either liquid or, particularly, solid form);
(b) an organic or an inorganic material; and/or
(c) exert a either a general or a specific pharmacological effect on an organism (e.g. a mammal such as a human) or exert no such effects.

Further, the core material may be either soluble or insoluble in the fluid used in the process of the invention. In particular embodiments of the invention, the core material is insoluble in the fluid used in the process of the invention (e.g. carbon dioxide).

In this respect, by “insoluble”, we mean that, under the supercritical conditions selected for the process (where T≧T_{c}, and P≧P_{c}), the core material has a solubility in the fluid, as measured by standard techniques, such as spectroscopic measurements (e.g. utraviolet-visible or infrared spectroscopy), of less than 1 mg/mL (e.g. less than 0.1 mg/mL, such as less than 10, 8, 5, 4 or, particularly, 3, 2 or 1 μg/mL). For example, the core material may have a solubility in the fluid of less than 10 μg/mL. When the fluid selected is carbon dioxide, the solubility of the core material may, for example, be determined at a pressure of 2000 psi (13.79 MPa) and a temperature of 40° C. (313.15 K).

Conversely, by “soluble”, we mean that, under the same conditions, the core material has a solubility in the fluid selected, as measured by the same techniques, of equal to or greater than the limit below which the material is deemed insoluble, for example equal to or greater than 1 μg/mL, such as equal to or greater than 2 or 3 μg/mL (e.g. equal to or greater than 4, 5, 8 or 10 μg/mL, such as equal to or greater than 0.1 or 1 mg/mL).

Due to the unique properties of supercritical fluids, such as supercritical carbon dioxide, they are most advantageously employed in the production of solid polymer matrices that incorporate core materials that are difficult to process using conventional (i.e. liquid) solvents, for example due to interactions between the core material and the solvent that either negatively affect the performance (e.g. biological activity) of the core material or render impossible or impractical the desired processing of the core material.

In this respect, core materials that may be mentioned include materials of biological origin, as well as materials derived from or structurally related to materials of biological origin. Thus, embodiments of the invention that may be mentioned include those in which the core material is a biologically active material (e.g. a biologically active material that is insoluble in the fluid used in the process of the invention (e.g. carbon dioxide)).

Biologically active materials that may be mentioned include pharmaceutical and veterinary products, i.e. pharmacologically active compounds that alter physiological processes with the aim of treating, preventing, curing, mitigating or diagnosing a disease.

Thus, in particular embodiments of the invention, the core material is a biologically active material and is one or more materials selected from:

(a) low molecular weight drugs,
(b) live or inactivated microorganisms;
(c) polysaccharides;
(d) nucleic acids;
(e) antibodies;
(f) proteins (including enzymes);
(g) peptides (including natural, semi-synthetic and synthetic peptides); and
(h) antigens.

By the term “low molecular weight drug” we mean a drug with a molecular weight of less than about 1000 Da. Examples of such drugs include, but are not limited to, acarbose, acetyl cysteine, acetylcholine chloride, acitretin, acyclovir, alatrofloxacin, albendazole, albuterol, alendronate, amantadine hydrochloride, ambenomium, amifostine, amiloride hydrochloride, aminocaproic acid, amiodarone, amlodipine, amphetamine, amphotericin B, aprotinin, aripiprazole, atenolol, atorvastatin, atovaquone, atracurium besylate, atropine, axitinib, azithromycin, azithromycin, aztreonam, bacitracin, baclofen, becalermin, beclomethsone, belladona, benezepril, benzonatate, bepridil hydrochloride, betamethasone, bicalutanide, bleomycin sulfate, budesonide, bupropion, busulphan, butenafine, calcifediol, calciprotiene, calcitriol, camptothecan, candesartan, capecitabine, capreomycin sulfate, capsaicin, carbamezepine, carboplatin, carotenes, cefamandole nafate, cefazolin sodium, cefepime hydrochloride, cefixime, cefonicid sodium, cefoperazone, cefotetan disodium, cefotoxime, cefoxitin sodium, ceftizoxime, ceftriaxone, cefuroxime axetil, celecoxib, cephalexin, cephapirin sodium, cerivistatin, cetrizine, chlorpheniramine, cholecalciferol, cidofovir, cilostazol, cimetidine, cinnarizine, ciprofloxacin, ciprofloxacin, cisapride, cisplatin, cladribine, clarithromycin, clemastine, clidinium bromide, clindamycin and clindamycin derivatives, clomiphene, clomipramine, clondronate, clopidrogel, codeine, coenzyme QI0, colistimethate sodium, colistin sulfate, cromalyn sodium, cyclobenzaprine, cyclosporine, cytarabine, danaproid, danazol, dantrolene, deforoxamine, dexchlopheniramine, diatrizoate megluamine and diatrizoate sodium, diclofenac, dicoumarol, dicyclomine, didanosine, digoxin, dihydro epiandrosterone, dihydroergotamine, dihydrotachysterol, dirithromycin, dirithromycin, donepezil, dopamine hydrochloride, doxacurium chloride, doxorubicin, editronate disodium, efavirenz, elanaprilat, enoxacin, ephedrine, epinephrine, eposartan, ergocalciferol, ergotamine, erythromycin, esmol hydrochloride, essential fatty acid sources, etodolac, etoposide, famiciclovir, famotidine, fenofibrate, fentanyl, fexofenadine, finasteride, flucanazole, fludarabine, fluoxetine, flurbiprofen, fluvastatin, foscarnet sodium, fosphenytion, frovatriptan, furazolidone, gabapentin, ganciclovir, gemfibrozil, gentamycin, glibenclamide, glipizide, glyburide, glycopyrolate, glymepride, grepafloxacin, griseofulvin, halofantrine, ibuprofen, iloperidone, indinavir sulfate, ipratropium bromide, irbesartan, irinotecan, isofosfamide, isosorbide dinitrate, isotreinoin, itraconazole, ivermectin, japanese lamivudine, ketoconazole, ketorolac, L-thryroxine, lamotrigine, lanosprazole, lapatinib, leflunomide, leucovorin calcium, levofloxacin, lincomycin and lincomycin derivatives, lisinopril, lobucavir, lomefloxacin, loperamide, loracarbef, loratadine, lovastatin, lutein, lycopene, mannitol, medroxyprogesterone, mefepristone, mefloquine, megesterol acetate, mephenzolate bromide, mesalmine, metformin hydrochloride, methadone, methanamine, methotrexate, methoxsalen, methscopolamine, metronidazole, metronidazole, metroprolol, mezocillin sodium, miconazole, midazolam, miglitol, minoxidil, mitoxantrone, mivacurium chloride, montelukast, nabumetone, nalbuphine, naratiptan, nedocromil sodium, nelfinavir, neostigmine bromide, neostigmine methyl sulfate, neutontin, nifedipine, nilsolidipine, nilutanide, nitrofurantoin, nizatidine, norfloxacin, ofloxacin, olanzapine, olpadronate, omeprazole, oprevelkin, osteradiol, oxaprozin, oxytocin, paclitaxel, paliperidone, pamidronate disodium, pancuronium bromide, paricalcitol, paroxetine, paroxetine, pazopanib, pefloxacin, pentamindine isethionate, pentazocine, pentostatin, pentoxifylline, periciclovir, phentolamine mesylate, phenylalanine, physostigmine salicylate, pioglitazone, piperacillin sodium, pizofetin, polymixin B sulfate, pralidoxine chloride, pravastatin, prednisolone, pregabalin, probucol, progesterone, propenthaline bromide, propofenone, pseudo-ephedrine, pyridostig mine, pyridostigmine bromide, rabeprazole, raloxifene, refocoxib, repaglinide, residronate, ribavarin, rifabutine, rifapentine, rimantadine hydrochloride, rimexolone, risperidone, ritanovir, rizatriptan, rosigiltazone, salmetrol xinafoate, saquinavir, sertraline, sibutramine, sildenafil citrate, simvastatin, sirolimus, solatol, sorafenib, sparfloxacin, spectinomycin, spironolactone, stavudine, streptozocin, sumatriptan, sunitinib, suxamethonium chloride, tacrine, tacrine hydrochloride, tacrolimus, tamoxifen, tamsulosin, targretin, tazarotene, telmisartan, teniposide, terbinafine, terbutaline sulfate, terzosin, tetrahydrocannabinol, thiopeta, tiagabine, ticarcillin, ticlidopine, tiludronate, timolol, tirofibran, tizanidine, topiramate, topotecan, toremifene, tramadol, trandolapril, tretinoin, trimetrexate gluconate, troglitazone, trospectinomycin, trovafloxacin, trovafloxacin, tubocurarine chloride, ubidecarenone, urea, valaciclovir, valsartan, valsartan, vancomycin, vecoronium bromide, venlafaxine, vertoporfin, vigabatrin, vinblastin, vincristine, vinorelbine, vitamin A, vitamin 812, vitamin D, vitamin E, vitamin K, warfarin sodium, zafirlukast, zalcitabine, zanamavir, zidovudine, zileuton, zolandronate, zolmitriptan, zolpidem, zopiclone, or a pharmaceutically acceptable salt thereof.

The core materials listed under categories (b) to (h) above which may be used in the invention typically have a molecular weight of from about 1 to about 300 kDa, more preferably from about 1 to about 150 kDa, more preferably from about 1 to 100 kDa and most preferably from about 1 to about 50 kDa. Illustrative examples of such core materials are as follows:

insulin (e.g. human insulin, insulin lispro, insulin procine, insulin NPH, insulin aspart, insulin glargine or insulin detemir),

antihemophilic factor (Factor VIII), such as porcine antihemophilic factor or, particularly, human antihemophilic factor, such as recombinant human antihemophilic factor,
Factor VII, Factor VIIa,
Factor IX,
growth hormones (such as bovine growth hormone or, particularly, human growth hormone, hGH, or recombinant hGH),
growth hormone releasing factor,
somatostatin,
glucagons,
parathyroid hormone (e.g. a recombinant parathyroid hormone, such as teriparatide),
calcitonin (e.g. human or salmon calcitonin);
interleukins (Ls), such as interleukin-2 (IL-2) or interleukin-3 (IL-3),
interleukin 1 receptor antagonist (IL-1 Ra),
interferons (IFNs), such as IFN alpha (e.g. IFN alpha 2a, PEGylated IFN alpha 2a, IFN alpha 2b, PEGylated IFN alpha 2b, human leukocyte IFN alpha (HuIFN-alpha-Le)), IFN beta (e.g. IFN beta 1a or IFN beta 1b) or IFN gamma (e.g. IFN gamma 1b),
vascular endothelium growth factor (VEGF),
anti-VEGF antibodies or fragments thereof (e.g. bevacizumab or ranibizumab),
erythropoietins (EPOs), such as epoetin alpha (e.g. Darbepoetin, Epocept, Epofit, Epogen, Epogin, Eprex, Nanokine or Procrit), epoetin beta (e.g. Recormon, NeoRecormon or methoxy polyethylene glycol-epoetin beta), epoetin delta (e.g. Dynepo), epoetin omega (e.g. Epomax) or epoetin zeta (e.g. Silapo or Retacrit),
heparin and its derivatives, such as heparin sodium or low molecular weight heparin (e.g. bemiparin, certoparin, dalteparin (e.g. daltaperin sodium), enoxaparin (e.g. enoxaprin sodium), nadroparin, parnaparin, reviparin or tinzaparin),
tissue plasminogen activator (t-PA), such as recombinant t-PA (e.g. alteplase, reteplase, tenecteplase or desmoteplase),
platelet derived growth factors (PDGFs), such as human PDGF,
cyclosporin A and analogs thereof (e.g. voclosporin),
oxytocin,
enkephalin,
tyrotropin releasing hormone,
vasopressin and vasopressin analogs,
catalase,
superoxide dismutase,
glatiramer acetate,
bone morphogenetic protein (BMP),
colony stimulating factors (CSFs), such as CSF1 (macrophage colony-stimulating factor), CSF2 (granulocyte macrophage colony-stimulating factor (GM-CSF), e.g. recombinant GM-CSF such as sargramostim) and CSF3 (granulocyte colony-stimulating factor (G-CSF), e.g. recombinant G-CSF such as filgrastim), tumor necrosis factors, such as tumour necrosis factor alpha (TNFα),
TNFα inhibitors, such as TNFR:Fc fusion proteins (e.g. etanercept) or anti-TNFα antibodies or fragments thereof (e.g. infliximab, adalimumab, certolizumab pegol or golimumab),
melanocyte stimulating hormone (MSH),
glucagon-like peptide-1 (GLP-1),
glucagon-like peptide-2 (GLP-2),
katacalcin,
cholecystekinin-12,
cholecystekinin-8,
exendin,
gonadoliberin-related peptide,
insulin-like protein,
leucine-enkephalin,
methionine-enkephalin,
leumorphin,
neurophysin,
copeptin,
neuropeptide Y,
neuropeptide AF,
PACAP-related peptide,
pancreatic hormone,
peptide YY,
urotensin,
intestinal peptide,
adrenocorticotropic peptide,
epidermal growth factor,
prolactin,
gastrin,
tetragastrin,
pentagastrin,
endorphins,
angiotensins,
thyrotropin releasing hormone,
heparinase,
alglucerase,
asparaginase,
cortocotropin,
denileukin diftitox,
dornase alpha,
streptokinase,
urokinase,
cosyntropin,
desmopressin,
octreotide acetate,
pramlintide,
sincalide,
enzymes,
glycoproteins,
antigens derived from or consisting of live or inactivated microorganisms (e.g. bacteria or viruses), such as BCG vaccine, cholera vaccine, encephalitis virus vaccine, hemophilus B conjugate vaccine, Hepatitis A virus vaccine inactivated, Hepatitis B virus vaccine inactivated, influenza virus vaccine, measles virus vaccine, meningococcal vaccine, mumps viral vaccine, plague vaccine, pneumococcal vaccine polyvalent, poliovirus vaccine live (OPV), poliovirus vaccine inactivated, rabies vaccine, rotavirus vaccine, small pox vaccine, typhoid vaccine live, varicella virus vaccine live, yellow fever vaccine, or combinations of such antigens or vaccines.

In particular embodiments of the invention, the core material is selected from the list consisting of: growth hormone (e.g. recombinant hGH); risperidone; paliperidone; aripiprazole; iloperidone; olanzapine; interferon alpha; interferon beta; glatiramer acetate; erythropoietin; anti-VEGF antibodies or fragments thereof (e.g. bevacizumab or ranibizumab); anti-TNFα antibodies or fragments thereof; Factor VII; Factor VIIa; Factor IX; BMP; and GLP-1, or the core material is an analogue of any of those materials.

Alternatively, the core material may be a natural or synthetic material capable of immobilising by absorption, interaction, reaction or otherwise naturally occurring or artificially introduced poisons, toxins or other biologically active agents.

In particular embodiments of the invention, the core material (e.g. any of the materials mentioned above) is provided in solid form, e.g. as particles or a powder. The size of the solid particles will depend on factors such as the nature and intended use of the core material. Typically the solid particles have a size of from about 1 nm to about 100 μm.

The amount of core material used in the process of the invention is not particularly limited and as the skilled person will appreciate the amount of active material will depend on a variety of factors including the nature and intended use of the material, as well as (if the material is a biologically active material such as defined above in respect of categories (a) to (h)) the intended dosage form and the intended dosage regimen.

Thus, embodiments of the invention that may be mentioned include those wherein the core material represents, for example, at least about 0.01% by weight of the product of the process of the invention, namely the solid polymer matrix containing the core material (e.g. the weight of core material is at least about 0.01% of the combined weight of the polymer and the core material). In such embodiments, the weight of core material may be, for example, from about 0.01% to about 95% of the combined weight of the polymer and the core material, such as from about 1 to about 50%, from about 2 to about 40%, from about 5% to about 30% or from about 10 to about 15 or 20% of the combined weight of the polymer and the core material.

### The (Supercritical) Fluid

The fluid used in the process of the present invention can be any fluid which may be brought into a supercritical state. As is known in the art, such fluids may be subjected to conditions of temperature and pressure up to a critical point at which the equilibrium line between liquid and vapour regions disappears. Supercritical fluids are characterised by properties which are both gas-like and liquid-like. In particular, the fluid density and solubility properties resemble those of liquids, whilst the viscosity, surface tension and fluid diffusion rate in any medium resemble those of a gas, giving gas-like penetration of the medium

Supercritical fluids which may be used include one or more (e.g. one) of: carbon dioxide; di-nitrogen oxide; carbon disulphide; aliphatic C₂₋₁₀ hydrocarbons such as ethane, propane, butane, pentane, hexane, ethene, propene, and halogenated derivatives thereof, such as carbon tetrafluoride, carbon tetrachloride, carbon monochloride trifluoride, fluoroform and chloroform; C₆₋₁₀ aromatics such as benzene, toluene and xylene; C₁₋₃ alcohols such as methanol and ethanol; sulphur halides such as sulphur hexafluoride; ammonia; xenon; and krypton.

Typically these fluids may be brought into supercritical conditions at a temperature of from about 0 to about 300° C. and a pressure of from about 7×10⁵ Nm⁻² to about 1×10⁸ Nm⁻², such as from about 12×10⁵ Nm⁻² to about 8×10⁷ Nm⁻² (7-1000 bar, such as 12-800 bar).

Critical temperatures and pressures of representative fluids are provided below.

| Fluid | Critical temperature, T_{c} (K) | Critical pressure, Tₚ (MPa) |
|---|---|---|
| Carbon dioxide | 304.1 | 7.38 |
| Water | 647.1 | 22.06 |
| Methane | 190.4 | 4.60 |
| Ethane | 305.3 | 4.87 |
| Propane | 369.8 | 4.25 |
| Ethene | 282.4 | 5.04 |
| Propene | 364.9 | 4.60 |
| Methanol | 512.6 | 8.09 |
| Ethanol | 513.9 | 6.14 |
| Acetone | 508.1 | 4.70 |

In particular embodiments of the invention, the fluid comprises or, more particularly, represents carbon dioxide. In such embodiments of the process of the invention, the conditions used in step (b) (to convert carbon dioxide to the supercritical state) are typically:
a temperature within the range from about 305 to about 320 K (approximately from about 32 to about 47° C.); and
a pressure within the range of about 7.4 to about 20.7 MPa (approximately from about 1073 to about 3000 psi).

It will be appreciated that the choice of fluid will depend on a variety of factors including the nature of the core material and the solid polymer. The nature of the solid polymer is particularly important in the selection of the supercritical fluid. Typically, the fluid should have both:
relatively high density in the supercritical phase (e.g. a density at the critical point of the fluid of at least twice the density at ambient temperature and pressure (e.g. 298 K and 1 atmosphere)); and
high solubility in the polymer.

The amount of supercritical fluid used in the process of the invention can vary within wide limits and may depend on factors such as the nature of the polymer and the nature of the reaction vessel.

### Process Step (b)

The mixing vessel used in step (b) of the process of the invention may be any vessel capable of withstanding the temperature and pressure conditions required to convert the selected fluid to the supercritical state. Thus, for example, the mixing vessel may be an autoclave or similar apparatus.

Mixing of the polymer, core material and supercritical fluid may be conveniently achieved by introducing the fluid into a mixing vessel containing a mixture of finely divided (e.g. powdered) polymer and core material, and then adjusting the pressure and/or temperature of the vessel such that the temperature is at or above T_{c} for the fluid and the pressure is at or above P_{c} for the fluid. In such embodiments of the invention, the processing aid (if used) may also be present in, or may be added to, the pre-mix of polymer and core material.

In certain embodiments of the invention, the mixture of polymer and core material may be prepared by mixing polymer (e.g. finely divided, such as powdered polymer) with a solution (in a conventional solvent) of core material and then freeze-drying the mixture. This method is convenient to use when, for example, it is desired to obtain a homogenous dispersion of particularly low quantities of core material (e.g. less than 1% core material by weight relative to the combined weight of the polymer and core material).

Optionally, mixing is continued whilst the fluid is in the supercritical state, for example by agitating (e.g. stirring) or pumping the contents of mixing vessel. In this respect, stirring may be conveniently carried out using a mechanical stirrer with which the mixing vessel may be equipped (see, for example, U.S. Pat. No. 5,548,004, the contents of which are incorporated herein by reference).

In particular embodiments of step (b) of the process of the invention, the supercritical fluid penetrates the polymer, thereby swelling and/or plasticizing the solid polymer and enabling dispersion of the core material throughout the polymer matrix.

Thus, embodiments of the invention that may be mentioned include those in which the solid polymer and the supercritical fluid are selected such that the polymer (i.e. at least one component of the solid polymer) is insoluble in the supercritical fluid.

In this respect, by “insoluble”, we mean that, under the supercritical conditions selected for the process (where T≧T_{c} and P≧P_{c}), the solid polymer has a solubility in the fluid, as measured by standard techniques, such as spectroscopic measurements (e.g. utraviolet-visible or infrared spectroscopy, of less than 1 mg/mL (e.g. less than 0.1 mg/mL, such as less than 10, 8, 5, 4 or, particularly, 3, 2 or 1 μg/mL). When the fluid selected is carbon dioxide, the solubility of the solid polymer may, for example, be determined at a pressure of 2000 psi (13.79 MPa) and a temperature of 40° C. (313.15 K).

Information on solubility of polymers in supercritical fluids may be found, for example, in Shine, Chapter 18: Polymers and Supercritical Fluids in Physical Properties of polymers Handbook, 249-256 (passim) (James E Mark ed. 1993), which is incorporated herein by reference.

The supercritical conditions achieved during process step (b) may be maintained for any suitable length of time, depending upon, for example, the nature of the polymer, core material and/or supercritical fluid and/or the temperature and pressure selected for the processing.

In particular embodiments of the invention, the supercritical conditions achieved during process step (b) are maintained for a time period of at least 1 minute (e.g. for a time period of from about 1, 2, 3, 4 or 5 to about 180 minutes, such as from about 10 or 20 to about 90 or 120 minutes or, particularly, from about 25 to 75 minutes, such as from about 30 minutes to about 60 minutes).

### The Processing Aid

The process of the invention may utilise one or more processing aids in order to achieve any one or more of the following objectives:
(i) a lower temperature and/or pressure at which the supercritical fluid plasticizes the polymer;
(ii) a lower viscosity for the (plasticized) mixture of polymer, core material and supercritical fluid; and
(iii) a more homogenous distribution of the core material throughout the polymer matrix produced by the process.

Achieving objectives (i) and/or (ii) above may provide advantages in respect of enabling better mixing of components under supercritical conditions and/or, particularly, better results (e.g. increased yield, smaller particle size, narrower particle size distribution, more spherical particle morphology) from spraying, during step (e), the plasticized mixture to form particles of polymer matrix containing core material.

Different processing aids may be used to achieve objectives (i) to (iii) above. For example, a polymer plasticizer may be used to achieve objective (i). Such a plasticizer may also achieve objective (ii), which can alternatively be achieved by an ampiphilic molecule, namely a molecule containing both polymer-philic and supercritical fluid-philic (e.g. CO₂-philic) regions.

Finally, objective (iii) may be achieved, for example, by use of a conventional solvent (i.e. a solvent that is liquid at ambient conditions, such as 298 K and 1 atmosphere pressure).

As will be evident from the following, certain processing aids may be polymeric materials. Such materials may therefore have dual functionality, i.e. they may serve as both (part of) the solid polymeric material and as (part of) the processing aid.

Processing aids which are suitable for use in the process of the present invention include conventional solvents, poloxamers, oligomers or polymers of fatty acids, fatty acid esters, hydroxy fatty acid esters, pyrolidones, polymeric pyrolidones, polyethers, medium and long chain triglycerides, phospholipids, derivatives thereof and mixtures thereof.

Conventional solvents that may be used as processing aids in the process of the present invention include aprotic organic solvents such as dimethylsulf oxide (DMSO) and acetone or alcohols such as ethanol.

Poloxamers are block copolymers of ethylene oxide and propylene oxide. They have the following general formula,

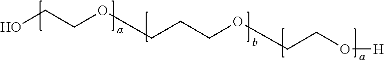

wherein each a is typically (independently) from 2 to 130 and b is typically from 15 to 67.

Several different types of poloxamer are available commercially, from suppliers such as BASF, and vary with respect to molecular weight and the proportions of ethylene oxide “a” units and propylene oxide “b” units. Poloxamers suitable for use in the subject invention typically have a molecular weight of from 2,500 to 18,000, for example from 7,000 to 15,000 Da. Particular examples of commercially available poloxamers include poloxamer 188, which structurally contains 80 “a” units and 27 “b” units, and has a molecular weight in the range 7680 to 9510 and poloxamer 407 which structurally contains 101 “a” units and 56 “b” units, and has a molecular weight in the range 9840 to 14600 (Handbook of Pharmaceutical Excipients, editor A. H. Kippe, third edition, Pharmaceutical Press, London, U K, 2000, which is incorporated herein by reference).

Fatty acids which are suitable for use as processing aids include linear and cyclic (preferably linear), saturated and unsaturated fatty acids comprising from 6 to 40, preferably from 9 to 30 and most preferably from 11 to 18 carbon atoms. The saturated fatty acids have the general formula CₙH₂ₙO₂, wherein n is from 7 to 40, preferably from 9 to 30 and most preferably from 11 to 18. The unsaturated fatty acids may have the formula CₙH₂ₙ₋₂O₂, or CₙH₂ₙ₋₄O₂ or CₙH₂ₙ₋₆O₂, wherein n is from 7 to 40, preferably from 9 to 30 and most preferably from 11 to 18. Unsaturated fatty acids with 4 or more double bonds may also be used. Optionally, the fatty acids may be hydroxylated (e.g. 12-hydroxy steric acid). The hydroxy group(s) may be further esterified with another fatty acid (i.e. fatty acid oligomers or polymers). Unsaturated fatty acids may be in the cis- or trans-configurations or mixtures of both configurations may be used.

Examples of preferred fatty acids include stearic acid, oleic acid, myristic acid, caprylic acid and capric acid. Oils containing these and any of the foregoing fatty acids may also be used as the processing aid, e.g. cotton seed oil, sesame oil and olive oil.

Suitable fatty acid derivatives (e.g. esters) include those that can be derived from the fatty acids and hydroxyl fatty acids defined above. Preferred fatty acid esters are mono-esters and di-esters of fatty acids, and derivatives thereof, such as polyethylene glycol (PEG) mono-esters and di-esters of fatty acids. Suitable PEGs include those having from 2 to 200 monomer units, preferably 4 to 100 monomer units, for example 10 to 15 monomer units. Examples include PEG stearate and PEG distearate, each available with varying PEG chain lengths e.g. polyoxyl 40 stearate (Crodet S40, Croda) and PEG-8 distearate (Lipopeg 4-DS, Adina).

A particular fatty acid ester that may be mentioned is Solutol® HS 15, which is available from BASF. Solutol® consists of polyglycol mono- and di-esters of 12-hydroxystearic acid and of about 30% by weight free polyethylene glycol and is an amphiphilic material having a hydrophilic-lipophilic balance of from about 14 to about 16.

Further examples of fatty acid derivatives include fatty acids esterified with polyoxyethylene sorbitan compounds, such as the “Tween” compounds (e.g. polyoxyethylene (20) sorbitan monooleate, also known as Tween 80) and fatty acids esterified with sorbitan compounds, such as the “Span” compounds (e.g. sorbitan monooleate, also known as Span 80).

Suitable pyrolidones include 2-pyrolidone, such as Soluphor® (BASF) and N-methyl-2-pyrrolidone.

Suitable polymeric pyrolidones include polyvinylpyrrolidone (e.g. Kollidon®).

Suitable polyethers include those comprising monomers comprising from 2 to 10 carbon atoms, preferably polyethylene glycols (PEGs) and polypropylene glycols (PPGs).

Suitable triglycerides include saturated and unsaturated medium and long chain mono-, di- and tri-glycerides.

Typically, medium chain mono-, di- and tri-glycerides have a formula (CH₂OR₁)(CH₂OR₂)(CH₂OR₃) wherein R₁, R₂ and R₃ are independently H or —C(O)(CH₂)ₙCH₃ (where n=6 to 8), provided that at not all R₁, R₂ and R₃=H. Preferable medium chain mono-, di- and tri-glycerides consist of a mixture of esters of saturated fatty acids mainly of capryilic acid and capric acid e.g. Crodamol GTC/C (Croda), Miglyol 810, Miglyol 812, Neobee M5.

Typically, long chain mono-, di- and tri-glycerides have a formula (CH₂OR₁)(CH₂OR₂)(CH₂OR₃) wherein R₁, R₂ and R₃ are independently H or —C(O)(CH₂)ₘCH₃ (where m=7 to 17), provided that at not all R₁, R₂ and R₃=H. A preferred long chain mono-, di- and tri-glyceride is Witepsol.

Particular processing aids that may be mentioned are amphiphilic processing aids. Suitable amphiphilic compounds typically have a hydrophilic-lipophilic balance (HLB) of from about 1 to about 50, preferably from about 5 to 30 and most preferably from about 12 to about 24. HLB values can be calculated using the method of Griffin published in Griffin W. C., 1954, Calculation of HLB values of non-ionic surfactants, J. Soc. Cosmet. Chem. 5, 249-256 and Griffin W. C., 1955, Calculation of HLB values of non-ionic surfactants, Am. Perf. Essent. Oil Rev., 26-29 (both of which are incorporated herein by reference).

In certain embodiments of the invention, the processing aid is not a conventional solvent and the process is carried out substantially in the absence (e.g. in the absence) of solvents other than the supercritical fluid.

In particular embodiments of the invention, the processing aid is a single component selected from the alternatives described above (e.g. a poloxamer, such as PL407).

The amount of processing aid used will depend upon various factors, including the nature of the solid polymer, the core material and/or the supercritical fluid. In this respect, the processing aid, if present, may represent, from about 0.2% to about 30%, such as from about 0.5% to about 15% (e.g. from about 8 to about 12%) by weight of the combined weight of the polymer, core material and processing aid.

### Process Steps (c) and (d)

Step (c) of the process of the present invention comprises the important steps of converting the fluid from supercritical to sub-critical state and then returning it to the supercritical state.

This “cycling” between super- and sub-critical states is effected without recovering the solid polymer matrix. By “without recovering”, we mean that the solid polymer matrix is not removed from the mixing vessel. Processes including at least one cycle as described in step (c) may provide various advantages as described below.

The conversion of the fluid from the supercritical to the sub-critical state (and then back again) may be achieved by varying the temperature and/or pressure applied to the mixing vessel. However, in particular embodiments of the invention, step (c) comprises the steps of:
(ia) converting the fluid in the vessel to a sub-critical state by reducing the pressure in said vessel to below P_{c}, and then
(iia) returning the fluid in the vessel to the supercritical state by increasing the pressure in the vessel.

In step (c) (e.g. step (ia) above of step (c)), the pressure may be reduced to a minimum of anywhere between ambient pressure (e.g. about 1 atmosphere) and 99% of P_{c} for the fluid used in the process, for example a minimum within the range of:
from about 2 atmospheres (about 0.2 MPa) to about 98% of P_{c} for the fluid used in the process;
from about 15 atmospheres (about 1.5 MPa) to about 97% of P_{c} for the fluid used in the process;
from about 35 atmospheres (about 3.5 MPa) to about 96% of P_{c} for the fluid used in the process;
from about 50 atmospheres (about 5.1 MPa) to about 95% of P_{c} for the fluid used in the process; or, particularly
from about 90 to about 97% (e.g. from about 92 to about 94%) of P_{c} for the fluid used in the process.

For example, when the fluid is carbon dioxide, the pressure in step (c) may be reduced to minimum pressure of within the range of about 6.5 to about 7.0 MPa (e.g. about 6.89 MPa (about 1000 psi)).

For the avoidance of doubt, the variations in pressure of the fluid described in respect of steps (ia) and (iia) above may be effected either with or, particularly, without temperature control (i.e. maintaining the temperature of the mixing vessel at the same temperature as prior to step (C)). As will be known to those skilled in the art, effecting pressure changes without controlling temperature will tend to lead to a drop in temperature when pressure is reduced, and an increase in temperature when pressure is increased.

In particular embodiments of the invention involving steps (ia) and (iia), the changes in pressure (either up or down) are effected:
over a period of time from about 1 to about 120 minutes (e.g. from about 2 to about 60 minutes, such as from about 3 to about 30, from about 4 to about 20 or from about 5 to about 15 minutes (e.g. about 10 minutes)); and/or
in the absence of active mixing (e.g. agitation such as stirring) of the contents of the mixing vessel.

Thus, for example, the period of time to complete each repetition of steps (i) and (ii) together (or (ia) and (iia) together) of step (c) may be anywhere from about 2 to about 240 minutes (e.g. from about 4 to about 120 minutes, such as from about 6 to about 60, from about 8 to about 40 or from about 10 to about 30 minutes (e.g. about 20 minutes)).

If repeated according to (optional) step (d) of the process of the invention, each repetition of the cycle of step (c) may be the same or different. In particular embodiments of the invention, each repetition is the same and may be in accordance with any of the embodiments outlined above.

Embodiments of the invention that may be mentioned include those in which step (d) comprises from 1 to 25, such as from 2 to 20, from 3 to 15 or, particularly, from 4 to 10 (e.g. 9) repetitions of the cycle of step (c).

When the process of the invention utilises a processing aid, embodiments corresponding to those outlined above apply equally to steps (c1) and (d1) of the process/

### Process Step (e)

Step (e) of the process of the invention comprises releasing the pressure in the vessel and recovering solid polymer matrix containing the core material.

The release of pressure may be effected using any suitable method known in the art and may be subsequent to or concurrent with ceasing of mixing of the contents of the mixing vessel.

In certain embodiments of the invention, the pressure is released by depressurisation of the mixing vessel, leaving the solid polymer matrix containing the core material in situ in the vessel (when returned to ambient pressure).

In alternative embodiments of the invention, the contents of mixing vessel are discharged (e.g. sprayed or extruded) through a nozzle or like orifice into a second vessel at lower pressure.

Discharging by spraying may be used to obtain particles (e.g. microparticles) of the solid polymer matrix containing the core material. If particularly rapid solidification of the polymer is required, or if it is desired to control the rate of egress of the fluid from the polymer matrix, then the second vessel into which the contents of the mixing vessel are discharged may contain a coolant (e.g. liquid nitrogen).

Discharging by extrusion may be conducted with or without a mold. In the absence of a mold, extrusion may, for example, be used to obtain the solid polymer matrix in the form of rods or fibres (depending upon the size and shape of the nozzle or orifice). A mold may be used to obtain different morphologies of the solid polymer matrix (e.g. monoliths or implants of a specific shape and/or size).

In such alternative embodiments of the invention, step (e) can be carried out using techniques for removing a gas, which are similar to spray drying techniques. Apparatus suitable for these techniques and the techniques themselves, are well known.

The conditions employed in step (e) can be manipulated to control of the size of the (micro)particles obtained. Typically, the blended mixture is removed from the mixing chamber (which is under supercritical conditions) into a separate container (which is not under supercritical conditions and may for example be under ambient conditions) through a nozzle or like orifice. The size of the aperture of the nozzle or orifice can optionally be controlled to control the size of the microparticles. Altering the conditions under which the polymer matrix is removed from the supercritical fluid or the rate of removal can also affect that particle size.

In step (e), the pressure can be released over a time period of fractions of a second to several days. However, in particular embodiments of the invention, the pressure is released rapidly (e.g. over a period of 5 minutes or less, such as 1 minute or less, 1 second or less, or, particularly, about 0.5 seconds or less).

### Additives

Additional components which may be used in the process of the invention include, but are not limited to, initiators, accelerators, hardeners, stabilisers, antioxidants, adhesion promoters, fillers and the like may be incorporated within the polymer. Markers and tags and the like may be incorporated to trace or detect administration or consumption of the composition according to known techniques.

If it is desired to introduce an adhesion promoter into the polymer composition, the promoter may be used to impregnate or coat particles of core material prior to introduction into the polymer composition, by means of simple mixing, spraying or other known coating techniques, in the presence or absence of a fluid as hereinbefore defined. Preferably coating is performed in conjunction with mixing with fluid as hereinbefore defined. For example, the adhesion promoter may be dissolved in fluid as hereinbefore defined and the solution contacted with core material particles as hereinbefore defined. Alternatively, the adhesion promoter may be introduced into the mixing vessel during the mixing step.

The core material may be treated prior to or during the incorporation into the polymer with any suitable materials adapted to enhance the performance or mechanical properties thereof. When the core material is biologically active, it may, for example, be treated with components such as binders adapted to promote adhesion to the polymer, dispersants to increase dispersion throughout the polymer and prevent aggregate formation, to increase dispersion as a suspension throughout a supercritical fluid, activators to accelerate any biofunctional effect in situ and the like.

Preferred adhesion promoters are those that are soluble in the fluid as hereinbefore defined. This means that any residual promoter that does not bind to the biologically active material or to the polymer is removed when the microparticles are removed from the supercritical fluid.

### The Product of the Process

The morphology of the solid polymer matrix containing core material that is the product of the process of the invention is not particularly limited. For example the core material may be distributed throughout the polymer matrix resembling a (co-)continuous morphology. The transition from coated or encapsulated particles to distributed mixtures may be merely a gradation of order of magnitude, whereby the microparticles may effectively comprise a plurality of core material particles independently coated with or encapsulated by a continuous phase of polymer matrix. This is conveniently termed particulate morphology.

If step (e) comprises depressurisation of the mixing vessel (leaving the solid polymer matrix containing the core material in situ in the vessel), the product of the process (which will typically have monolithic morphology at the macroscopic scale) may be converted to (micro)particulate form by breaking up (e.g. grinding or milling) that product.

In certain embodiments of the invention in which step (e) involves a release of pressure by spraying the contents of the mixing vessel into another vessel, the microparticles produced using the process of the invention have a mean particle size expressed as the volume mean diameter (VMD) of from about 2, 3, 4, 5, 8 or 10 to about 500 μm, such as from about 20 to about 200 or 250 μm, from about 25 to about 150 μm, from about 30 to 100 μm, or, particularly, from about 35 to about 80 μm. The volume mean diameter of the microparticles can be measured by techniques well known in the art such as laser diffraction.

In more particular embodiments of the invention, no more than 10% of the microparticles have a diameter (D₁₀%) less than the lower limit of each of the size ranges quoted above respectively and at least 90% of the particles have a diameter (D₉₀%) that does not exceed the upper limit of each of the size ranges quoted above respectively.

### EMBODIMENTS

The following, numbered passages illustrate specific embodiments of the invention.
(1) A process for preparing a solid polymer matrix containing a core material, said process comprising the steps of:
(a) providing a solid polymer, a core material and a fluid that is capable of existing in the supercritical state;
(b) in a mixing vessel, mixing the polymer, core material and fluid at
   a temperature at or above T_{c} and
   a pressure at or above P_{c},
   such that the fluid is in the supercritical state, wherein T_{c} and P_{c} are the critical temperature and the critical pressure, respectively, for the fluid;
(c) without recovering the solid polymer matrix,
   (i) converting the fluid in the vessel to a sub-critical state by reducing the pressure in said vessel to below ID, and/or reducing the temperature in said vessel to below T_{c}, and then
   (ii) returning the fluid in the vessel to the supercritical state by increasing the pressure and/or the temperature in the vessel;
(d) optionally repeating step (c) one or more times; and
(e) releasing the pressure in the vessel and recovering solid polymer matrix containing the core material,
   provided that the core material does not comprise any of gonadotropin releasing hormone (GnRH), a GnRH agonist and a GnRH antagonist.
(2) A process according to paragraph (1), wherein the process comprises the steps of:
(a1) providing a solid polymer, a core material, a processing aid and a fluid that is capable of existing in the supercritical state;
(b1) in a mixing vessel, mixing the polymer, core material, processing aid and fluid at
   a temperature at or above T_{c} and
   a pressure at or above P_{c},
   such that the fluid is in the supercritical state, wherein T_{c} and P_{c} are the critical temperature and the critical pressure, respectively, for the fluid;
(c1) without recovering the solid polymer matrix,
   (i) converting the fluid in the vessel to a sub-critical state by reducing the pressure in said vessel to below P_{c} and/or reducing the temperature in said vessel to below T_{c}, and then
   (ii) returning the fluid in the vessel to the supercritical state by increasing the pressure and/or the temperature in the vessel;
(d1) optionally repeating step (c) one or more times; and
(e1) releasing the pressure in the vessel and recovering solid polymer matrix containing the core material,
   provided that the core material does not comprise any of gonadotropin releasing hormone (GnRH), a GnRH agonist and a GnRH antagonist.
(3) A process according to paragraph (1) or paragraph (2), wherein the solid polymer comprises one or more polymers selected from homopolymers, block and random copolymers and polymeric blends, any of which may be straight chain, branched or cross-linked.
(4) A process according to any one of paragraphs (1) to (3), wherein the solid polymer comprises a synthetic biodegradable polyester.
(5) A process according to any one of paragraphs (1) to (4), wherein the solid polymer comprises one or more polymers selected from:
   PHAs (e.g. PLA, PGA, PLGA, copolymers of lactic and glycolic acid with poly(ethyleneglycol), PCL or PHB);
   poly (ether esters) (e.g. poly(p-dioxanone));
   polymers of diacids and diols (e.g. poly(propylene fumarate) or poly(alkylene oxalates)); and
   poly(ether ester) multiblock copolymers (e.g. polymers based upon poly(ethylene glycol) and poly(butylene terephthalate)).
(6) A process according to any one of paragraphs (1) to (5), wherein the solid polymer comprises PLGA, PLA, or a combination of PLA and PLGA.
(7) A process according to any one of paragraphs (1) to (6), wherein the solid polymer is a single polymer (e.g. PLA, PGA or PLGA).
(8) A process according to any one of paragraphs (1) to (7), wherein the inherent viscosity of the solid polymer is from about 0.05 to about 0.15 dL/g (such as about 0.10 dL/g).
(9) A process according to any one of paragraphs (1) to (8), wherein the solid polymer is a mixture of two or more of:
   (i) a first polyester (e.g. PLGA);
   (ii) a second polyester (e.g. PLA or PGA); and
   (iii) a polyether (e.g. PEG or a block copolymer of ethylene glycol and propylene glycol).
(10) A process according to paragraph (9), wherein the polyether is a block copolymer of ethylene glycol and propylene glycol and has the following formula,

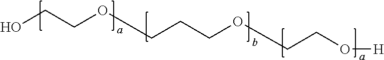

wherein each a is independently from 2 to 130 and b is from 15 to 67.
(11) A process according to paragraph (9) or paragraph (10), wherein the polyether is poloxamer PL407.
(12) A process according to any one of paragraphs (9) to (11), wherein the solid polymer is a mixture of PLGA and a polyether, or a mixture of PLGA, PLA and a polyether.
(13) A process according to any one of paragraphs (1) to (12), wherein any PLGA present in the solid polymer has a molar ratio of lactic acid:glycolic acid of from about 75:25 to about 25:75 (e.g. about 50:50).
(14) A process according to any one of paragraphs (1) to (12), wherein the solid polymer comprises both PLGA and PLA and, optionally, a polyether as defined in any of paragraphs (8) to (11) and the ratio by weight of PLGA:PLA is from about 90:10 to about 40:60 (e.g. from about 85:15 to about 50:50, such as from about 75:25 to about 60:40).
(15) A process according to paragraph (14), wherein a polyether is present at from about 5 to about 25% (e.g. from about 8 to about 15% or, particularly, from about 10 to about 12%) of the combined weight of PLGA and PLA.
(16) A process according to any one of paragraphs (1) to (15), wherein the solid polymer represents from about 5 to about 99.9% by weight (e.g. from about 25 to about 97, 98 or 99%, such as from about 45 to about 93%) of the combined weight of the solid polymer and the core material.
(17) A process according to any one of paragraphs (1) to (16), wherein the core material is insoluble in the fluid used under the supercritical conditions selected for the process (for example, wherein the core material has a solubility in the fluid of less than 10 μg/mL).
(18) A process according to any one of paragraphs (1) to (17), wherein the core material is a biologically active material.
(19) A process according to any one of paragraphs (1) to (18), wherein the core material is a biologically active material and is one or more materials selected from:
   (a) low molecular weight drugs,
   (b) live or inactivated microorganisms;
   (c) polysaccharides;
   (d) nucleic acids;
   (e) antibodies;
   (f) proteins;
   (g) peptides; and
   (h) antigens.
(20) A process according to any one of paragraphs (1) to (19), wherein the core material is selected from one or more of acarbose, acetyl cysteine, acetylcholine chloride, acitretin, acyclovir, alatrofloxacin, albendazole, albuterol, alendronate, amantadine hydrochloride, ambenomium, amifostine, amiloride hydrochloride, aminocaproic acid, amiodarone, amlodipine, amphetamine, amphotericin B, aprotinin, aripiprazole, atenolol, atorvastatin, atovaquone, atracurium besylate, atropine, axitinib, azithromycin, azithromycin, aztreonam, bacitracin, baclofen, becalermin, beclomethsone, belladona, benezepril, benzonatate, bepridil hydrochloride, betamethasone, bicalutanide, bleomycin sulfate, budesonide, bupropion, busulphan, butenafine, calcifediol, calciprotiene, calcitriol, camptothecan, candesartan, capecitabine, capreomycin sulfate, capsaicin, carbamezepine, carboplatin, carotenes, cefamandole nafate, cefazolin sodium, cefepime hydrochloride, cefixime, cefonicid sodium, cefoperazone, cefotetan disodium, cefotoxime, cefoxitin sodium, ceftizoxime, ceftriaxone, cefuroxime axetil, celecoxib, cephalexin, cephapirin sodium, cerivistatin, cetrizine, chlorpheniramine, cholecalciferol, cidofovir, cilostazol, cimetidine, cinnarizine, ciprofloxacin, ciprofloxacin, cisapride, cisplatin, cladribine, clarithromycin, clemastine, clidinium bromide, clindamycin and clindamycin derivatives, clomiphene, clomipramine, clondronate, clopidrogel, codeine, coenzyme QI0, colistimethate sodium, colistin sulfate, cromalyn sodium, cyclobenzaprine, cyclosporine, cytarabine, danaproid, danazol, dantrolene, deforoxamine, dexchlopheniramine, diatrizoate megluamine and diatrizoate sodium, diclofenac, dicoumarol, dicyclomine, didanosine, digoxin, dihydro epiandrosterone, dihydroergotamine, dihydrotachysterol, dirithromycin, dirithromycin, donepezil, dopamine hydrochloride, doxacurium chloride, doxorubicin, editronate disodium, efavirenz, elanaprilat, enoxacin, ephedrine, epinephrine, eposartan, ergocalciferol, ergotamine, erythromycin, esmol hydrochloride, essential fatty acid sources, etodolac, etoposide, famiciclovir, famotidine, fenofibrate, fentanyl, fexofenadine, finasteride, flucanazole, fludarabine, fluoxetine, flurbiprofen, fluvastatin, foscarnet sodium, fosphenytion, frovatriptan, furazolidone, gabapentin, ganciclovir, gemfibrozil, gentamycin, glibenclamide, glipizide, glyburide, glycopyrolate, glymepride, grepafloxacin, griseofulvin, halofantrine, ibuprofen, iloperidone, indinavir sulfate, ipratropium bromide, irbesartan, irinotecan, isofosfamide, isosorbide dinitrate, isotreinoin, itraconazole, ivermectin, japanese lamivudine, ketoconazole, ketorolac, L-thryroxine, lamotrigine, lanosprazole, lapatinib, leflunomide, leucovorin calcium, levofloxacin, lincomycin and lincomycin derivatives, lisinopril, lobucavir, lomefloxacin, loperamide, loracarbef, loratadine, lovastatin, lutein, lycopene, mannitol, medroxyprogesterone, mefepristone, mefloquine, megesterol acetate, mephenzolate bromide, mesalmine, metformin hydrochloride, methadone, methanamine, methotrexate, methoxsalen, methscopolamine, metronidazole, metronidazole, metroprolol, mezocillin sodium, miconazole, midazolam, miglitol, minoxidil, mitoxantrone, mivacurium chloride, montelukast, nabumetone, nalbuphine, naratiptan, nedocromil sodium, nelfinavir, neostigmine bromide, neostigmine methyl sulfate, neutontin, nifedipine, nilsolidipine, nilutanide, nitrofurantoin, nizatidine, norfloxacin, ofloxacin, olanzapine, olpadronate, omeprazole, oprevelkin, osteradiol, oxaprozin, oxytocin, paclitaxel, paliperidone, pamidronate disodium, pancuronium bromide, paricalcitol, paroxetine, paroxetine, pazopanib, pefloxacin, pentamindine isethionate, pentazocine, pentostatin, pentoxifylline, periciclovir, phentolamine mesylate, phenylalanine, physostigmine salicylate, pioglitazone, piperacillin sodium, pizofetin, polymixin B sulfate, pralidoxine chloride, pravastatin, prednisolone, pregabalin, probucol, progesterone, propenthaline bromide, propofenone, pseudo-ephedrine, pyridostigmine, pyridostigmine bromide, rabeprazole, raloxifene, refocoxib, repaglinide, residronate, ribavarin, rifabutine, rifapentine, rimantadine hydrochloride, rimexolone, risperidone, ritanovir, rizatriptan, rosigiltazone, salmetrol xinafoate, saquinavir, sertraline, sibutramine, sildenafil citrate, simvastatin, sirolimus, solatol, sorafenib, sparfloxacin, spectinomycin, spironolactone, stavudine, streptozocin, sumatriptan, sunitinib, suxamethonium chloride, tacrine, tacrine hydrochloride, tacrolimus, tamoxifen, tamsulosin, targretin, tazarotene, telmisartan, teniposide, terbinafine, terbutaline sulfate, terzosin, tetrahydrocannabinol, thiopeta, tiagabine, ticarcillin, ticlidopine, tiludronate, timolol, tirofibran, tizanidine, topiramate, topotecan, toremifene, tramadol, trandolapril, tretinoin, trimetrexate gluconate, troglitazone, trospectinomycin, trovafloxacin, trovafloxacin, tubocurarine chloride, ubidecarenone, urea, valaciclovir, valsartan, valsartan, vancomycin, vecoronium bromide, venlafaxine, vertoporfin, vigabatrin, vinblastin, vincristine, vinorelbine, vitamin A, vitamin B12, vitamin D, vitamin E, vitamin K, warfarin sodium, zafirlukast, zalcitabine, zanamavir, zidovudine, zileuton, zolandronate, zolmitriptan, zolpidem, zopiclone, or a pharmaceutically acceptable salt thereof.
(21) A process according to any one of paragraphs (1) to (19), wherein the core material is selected from one or more of
   insulin (e.g. human insulin, insulin lispro, insulin procine, insulin NPH, insulin aspart, insulin glargine or insulin detemir),
   antihemophilic factor (Factor VIII), such as porcine antihemophilic factor or, particularly, human antihemophilic factor, such as recombinant human antihemophilic factor,
   Factor VII, Factor Vila,
   Factor IX,
   growth hormones (such as bovine growth hormone or, particularly, human growth
   hormone, hGH, or recombinant hGH),
   growth hormone releasing factor,
   somatostatin,
   glucagons,
   parathyroid hormone (e.g. a recombinant parathyroid hormone, such as teriparatide),
   calcitonin (e.g. human or salmon calcitonin);
   interleukins (ILs), such as interleukin-2 (IL-2) or interleukin-3 (IL-3),
   interleukin 1 receptor antagonist (IL-1 Ra),
   interferons (IFNs), such as IFN alpha (e.g. IFN alpha 2a, PEGylated IFN alpha 2a, IFN alpha 2b, PEGylated IFN alpha 2b, human leukocyte IFN alpha (HuIFN-alpha-Le)), IFN beta (e.g. IFN beta 1a or IFN beta 1b) or IFN gamma (e.g. IFN gamma 1b),
   vascular endothelium growth factor (VEGF),
   anti-VEGF antibodies or fragments thereof (e.g. bevacizumab or ranibizumab),
   erythropoietins (EPOs), such as epoetin alpha (e.g. Darbepoetin, Epocept, Epofit, Epogen, Epogin, Eprex, Nanokine or Procrit), epoetin beta (e.g. Recormon, NeoRecormon or methoxy polyethylene glycol-epoetin beta), epoetin delta (e.g. Dynepo), epoetin omega (e.g. Epomax) or epoetin zeta (e.g. Silapo or Retacrit),
   heparin and its derivatives, such as heparin sodium or low molecular weight heparin (e.g. bemiparin, certoparin, dalteparin (e.g. daltaperin sodium), enoxaparin (e.g. enoxaprin sodium), nadroparin, parnaparin, reviparin or tinzaparin),
   tissue plasminogen activator (t-PA), such as recombinant t-PA (e.g. alteplase, reteplase, tenecteplase or desmoteplase),
   platelet derived growth factors (PDGFs), such as human PDGF,
   cyclosporin A and analogs thereof (e.g. voclosporin),
   oxytocin,
   enkephalin,
   tyrotropin releasing hormone,
   vasopressin and vasopressin analogs,
   catalase,
   superoxide dismutase,
   glatiramer acetate,
   bone morphogenetic protein (BMP),
   colony stimulating factors (CSFs), such as CSF1 (macrophage colony-stimulating factor), CSF2 (granulocyte macrophage colony-stimulating factor (GM-CSF), e.g. recombinant GM-CSF such as sargramostim) and CSF3 (granulocyte colony-stimulating factor (G-CSF), e.g. recombinant G-CSF such as filgrastim),
   tumor necrosis factors, such as tumour necrosis factor alpha (TNFα),
   TNFα inhibitors, such as TNFR: Fc fusion proteins (e.g. etanercept) or anti-TNFα antibodies or fragments thereof (e.g. infliximab, adalimumab, certolizumab pegol or golimumab),
   melanocyte stimulating hormone (MSH),
   glucagon-like peptide-1 (GLP-1),
   glucagon-like peptide-2 (GLP-2),
   katacalcin,
   cholecystekinin-12,
   cholecystekinin-8,
   exendin,
   gonadoliberin-related peptide,
   insulin-like protein,
   leucine-enkephalin,
   methionine-enkephalin,
   leumorphin,
   neurophysin,
   copeptin,
   neuropeptide Y,
   neuropeptide AF,
   PACAP-related peptide,
   pancreatic hormone,
   peptide YY,
   urotensin,
   intestinal peptide,
   adrenocorticotropic peptide,
   epidermal growth factor,
   prolactin,
   gastrin,
   tetragastrin,
   pentagastrin,
   endorphins,
   angiotensins,
   thyrotropin releasing hormone,
   heparinase,
   alglucerase,
   asparaginase,
   cortocotropin,
   denileukin diftitox,
   dornase alpha,
   streptokinase,
   urokinase,
   cosyntropin,
   desmopressin,
   octreotide acetate,
   pramlintide,
   sincalide,
   enzymes,
   glycoproteins,
   antigens derived from or consisting of live or inactivated microorganisms (e.g. bacteria or viruses), such as BCG vaccine, cholera vaccine, encephalitis virus vaccine, hemophilus B conjugate vaccine, Hepatitis A virus vaccine inactivated, Hepatitis B virus vaccine inactivated, influenza virus vaccine, measles virus vaccine, meningococcal vaccine, mumps viral vaccine, plague vaccine, pneumococcal vaccine polyvalent, poliovirus vaccine live (OPV), poliovirus vaccine inactivated, rabies vaccine, rotavirus vaccine, small pox vaccine, typhoid vaccine live, varicella virus vaccine live, yellow fever vaccine, or combinations of such antigens or vaccines.
(22) A process according to any one of paragraphs (1) to (19), wherein the core material is a growth hormone (e.g. recombinant hGH), or an analogue thereof.
(23) A process according to any one of paragraphs (1) to (19), wherein the core material is selected from the list consisting of risperidone; paliperidone; aripiprazole; iloperidone; olanzapine; interferon alpha; interferon beta; glatiramer acetate; erythropoietin; anti-VEGF antibodies or fragments thereof (e.g. bevacizumab or ranibizumab); anti-TNFα antibodies or fragments thereof; Factor VII; Factor Vila; Factor IX; BMP; and GLP-1, or the core material is an analogue of any of those materials.
(24) A process according to any one of paragraphs (1) to (23), wherein the core material represents from about 0.01% to about 95% (e.g. from about 1 to about 50%, from about 2 to about 40%, from about 5% to about 30% or from about 10 to about 15 or 20%) of the combined weight of the solid polymer and the core material.
(25) A process according to any one of paragraphs (1) to (24), wherein the fluid is carbon dioxide.
(26) A process according to paragraph (25), wherein the conditions used in step (b) to convert carbon dioxide to the supercritical state are:
   a temperature within the range from about 305 to about 320 K; and
   a pressure within the range of about 7.4 to about 20.7 MPa.
(27) A process according to any one of paragraphs (1) to (26), wherein at least one component of the solid polymer is insoluble in the fluid under the supercritical conditions selected for the process.
(28) A process according to any one of paragraphs (1) to (27), wherein the supercritical conditions achieved during process step (b) are maintained for a time period of from about 1, 2, 3, 4 or 5 to about 180 minutes (e.g. from about 10 or 20 to about 90 or 120 minutes or, particularly, from about 25 to 75 minutes, such as from about 30 minutes to about 60 minutes).
(29) A process according to any one of paragraphs (1) to (28), wherein during step (b) the contents of the mixing vessel are agitated (e.g. by stirring) whilst the fluid is in the supercritical state.
(30) A process according to any one of paragraphs (2) to (29), wherein the processing aid, if used, is selected from conventional solvents, poloxamers, oligomers or polymers of fatty acids, fatty acid esters, hydroxy fatty acid esters, pyrolidones, polymeric pyrolidones, polyethers, medium and long chain triglycerides, phospholipids, derivatives thereof and mixtures thereof.
(31) A process according to paragraph (30), wherein the processing aid is selected from one or more of aprotic organic solvents (such as DMSO or acetone), alcohols such as ethanol, polyethers as defined in any one of paragraphs (9) to (11), polyglycol mono- and di-esters of 12-hydroxystearic acid and polyethylene glycol.
(32) A process according to paragraph (30), wherein the processing aid is a mixture comprising polyglycol mono- and di-esters of 12-hydroxystearic acid and about 30% by weight free polyethylene glycol.
(33) A process according to any one of paragraphs (30) to (32), wherein the processing aid represents from about 0.2% to about 30% (e.g. from about 0.5% to about 15% or from about 8 to about 12%) by weight of the combined weight of the polymer, core material and processing aid.
(34) A process according to any one of paragraphs (1) to (33), wherein step (c) comprises the steps of:
   (ia) converting the fluid in the vessel to a sub-critical state by reducing the pressure in said vessel to below P_{c}, and then
   (iia) returning the fluid in the vessel to the supercritical state by increasing the pressure in the vessel.
(35) A process according to any one of paragraphs (1) to (34), wherein in step (c) the pressure is reduced to a minimum within the range of from about 0.2 MPa to 98% of P_{c} for the fluid used in the process (e.g. from 1.5, 3.5 or 5.1 MPa to 95, 96 or 97% of P_{c} for the fluid used in the process, such as from about 90 to about 97% or from about 92 to about 94% of P_{c} for the fluid used in the process).
(36) A process according to any one of paragraphs (1) to (35), wherein when the fluid is carbon dioxide and the pressure in step (c) is reduced to minimum within the range of about 6.5 to about 7.0 MPa (e.g. about 6.89 MPa (about 1000 psi)).
(37) A process according to any one of paragraphs (1) to (36), wherein step (c) is effected in the absence of active mixing (e.g. agitation such as stirring) of the contents of the mixing vessel.
(38) A process according to any one of paragraphs (1) to (37), wherein the period of time to complete each repetition of steps (i) and (ii) together (or (ia) and (iia) together) of step (c) is from about 2 to about 240 minutes (such as from about 4 to about 120 minutes, from about 6 to about 60, from about 8 to about 40 or from about 10 to about 30 minutes (e.g. about 20 minutes)).
(39) A process according to any one of paragraphs (1) to (38), wherein, if repeated according to step (d), each repetition of the cycle of step (c) is the same.
(40) A process according to any one of paragraphs (1) to (39), wherein step (d) comprises from 1 to 25 (such as from 2 to 20, from 3 to 15 or, particularly, from 4 to 10 (e.g. 9)) repetitions of the cycle of step (c).
(41) A process according to any one of paragraphs (1) to (40), wherein in step (e) the pressure is released by depressurisation of the mixing vessel, leaving the solid polymer matrix containing the core material in situ in the vessel.
(42) A process according to any one of paragraphs (1) to (40), wherein in step (e) the contents of mixing vessel are discharged (e.g. sprayed) through a nozzle or like orifice into a second vessel at lower pressure.
(43) A process according to any one of paragraphs (1) to (42), wherein the solid polymer comprises two or more polymers that are solid and the product of the process comprises a true blend of those polymers.
(44) A solid polymer matrix containing a core material that is obtainable by (or is obtained by) a process according to any one of paragraphs (1) to (43), provided that the core material does not comprise any of gonadotropin releasing hormone (GnRH), a GnRH agonist and a GnRH antagonist.
(45) A process for preparing a pharmaceutical composition comprising a solid polymer matrix that contains a core material, wherein the core material is a biologically active material, provided that the core material does not comprise any of gonadotropin releasing hormone (GnRH), a GnRH agonist and a GnRH antagonist, said process comprising a process according to any one of paragraphs (1) to (43), followed by a step of formulating the solid polymer matrix for pharmaceutical use.

### FIGURES

FIG. 1: illustration of the cumulative release of degarelix from a polymer formulation prepared with a processing aid (DMSO) and either with (upper line) or without (lower line) the use of pressure cycling. As can be seen from the graph of FIG. 1, the effect of 10 pressure cycles was a decrease of approximately 15% in the initial burst release of degarelix from the formulation.

FIG. 2: illustration of the cumulative release of degarelix from a polymer formulation prepared without a processing aid but either with (upper line) or without (lower line) the use of pressure cycling. As can be seen from the graph of FIG. 2, the effect of 10 pressure cycles was a decrease of approximately 3% in the initial burst release of degarelix from the formulation.

FIG. 3: illustration of the cumulative release of BSA from a polymer formulation prepared with a processing aid (Poloxamer 407) and either with (upper line) or without (lower line) the use of pressure cycling. As can be seen from the graph of FIG. 3, the effect of 10 pressure cycles was a decrease of approximately 10% in the initial burst release of BSA from the formulation.

Processes of the invention may possess the advantage that they provide a solid polymer matrix containing a core material, wherein release of the core material from the matrix (e.g. either release into a liquid in vitro or release in vivo) demonstrates an enhanced profile relative to solid polymer matrices containing core material as made by known processes that utilise supercritical fluids. In this respect, and relative to such known solid polymer matrices, the release profile of the core material from the polymer matrix prepared according to the process of the present invention may demonstrate, for example:
a reduced “burst effect”; and/or
more sustained release (i.e. it may take longer to release all of the core material in the matrix).

Processes of the invention may also (or alternatively) possess the advantage that, compared to known processes utilising supercritical fluids, they provide the product:
by a more convenient or efficient process (e.g. a process utilising fewer steps, energy and/or materials to reach a product having the desired release profile);
in higher yield; and/or
in more uniform or otherwise more advantageous morphology (including a more uniform distribution of the core material throughout the polymer matrix and/or, when the product is obtained in particulate form, a more uniform distribution of particle size and/or a smaller particle size).

### Testing Methods

### Particle Size Measurements

Measurements realting to particle size (e.g. VMD, d90, d50 and d10) were obtained by standard techniques (laser diffraction). The laser diffraction measurements were conducted at 6 bar air pressure and ambient (room) temperature, and were conducted on samples comprising particles dispersed in an aqueous solution of polyoxyethylene (20) sorbitan monolaurate (otherwise known as Polysorbate 20 or Tween 20).

### In Vitro Release Testing—Degarelix

In-vitro release of microparticles is conducted with a manitol/acetate buffer solution at pH 4. 1 mL of this buffer is added to 10 mg of microparticles in a 1.5 mL Eppendorf tube and rotated at 10 rpm in an incubator at 37° C. Each sample is analysed in triplicate. At a time point a sample is removed and centrifuged at 8000 rpm for 3 min. 800 μL of supernatant is removed which is further centifruged at 13000 rpm for 3 min to acquire a 200 μL sample for HPLC analysis. The supernatant is replaced with fresh buffer and the sample placed back in the incubator.

Loading is calculated separately from the release samples using an anti-solvent precipitation method. A 25 mg sample is weighed out into a 25 mL volumetric flask. 1 mL of acetone is added to the volumetric flask to dissolve the microparticles. Once dissolved, the volumetric flask is topped up with water (approximately 24 mL), precipitating the polymer. A 1 mL sample of the supernatant is taken and centrifuged at 13000 rpm for 3 min. From this, a 200 μL sample is taken and analysed by HPLC. The loading determination method is carried out in triplicate and an average is taken.

### In Vitro Peptide Quantification-End of Study—Degarelix

In order to quantify the loading of Degarelix within the microparticle release study, the remaining polymer component of the formulation is substantially or totally removed from the peptide. A DCM/Acetone Extraction method is used to achieve this. The polymer is dissolved away from peptide by repeated washes with a DCM/Acetone (2:1) solution. The peptide is then dried and dissolved in H₂O for HPLC analysis. This method relies on the peptide being insoluble in the organic phase.

### In Vitro Release Testing—BSA

In-vitro release of microparticles is conducted with a HEPES buffer solution at pH 7.4. 1 mL of this buffer is added to 10 mg of microparticles in a 1.5 mL Eppendorf tube and rotated at 10 rpm in an incubator at 37° C. Each sample is analysed in triplicate. At a time point a sample is removed and centrifuged at 8000 rpm for 3 min. 800 μL of supernatant is removed which is further centifruged at 13000 rpm for 3 min to acquire a 200 μL sample for HPLC analysis. The supernatant is replaced with fresh buffer and the sample placed back in the incubator.

Loading is calculated separately from the release samples using an DCM/Acetone extraction method. A 10 mg sample is weighted out in triplicate into Eppendorfs. 1 mL of DCM/acetone solution is added to each Eppendorf to dissolve the PLGA. After being inverted several times the Eppendorfs are centrifuged at 13000 rpm for 3 min. From each Eppendorf 800 μL is taken and replaced with fresh DCM/acetone solution. This is repeated 3 times with each Eppendorf. On the last repeat, as much of the supernatant is removed as possible without disturbing the solid peptide. The Eppendorfs are left in a fume hood until all of the solvent has evaporated and the remaining peptide is dry (approximately 24 hrs). The peptide is dissolved in 1 mL of phosphate buffer and analysed via HPLC.

In vitro Peptide Quantification-End of Study—BSA

In order to quantify the loading of BSA within the microparticle release study, the remaining polymer component of the formulation is substantially or totally removed from the peptide. The DCM/Acetone Extraction method (detailed above in connection with Degarelix) is used to achieve this. The polymer is dissolved away from peptide by repeated washes with a DCM/Acetone (2:1) solution. The peptide is then dried and dissolved in phosphate buffer for HPLC analysis. This method relies on the peptide being insoluble in the organic phase.

### Worked Examples

The invention is illustrated by the following Examples.

### Reference Example 1a

### Degarelix Processed with DMSO and Pressure Cycling

Although this example illustrates the principles of the process of the invention, it does not fall within the scope of the attached claims because degarelix is a GnRH antagonist.

### Method

PLGA 75:25 (M_{w} 8 kDa, measured in THF relative to PS standards, 1.89 g) was mixed with Degarelix (0.21 g, 10 wt. %) by shaking/inverting the weighting vial containing both components. This mixture was loaded in to a supercritical fluid PGSS processing apparatus (see, for example, J. Pharm. Sci., 93(4), 1083-1090 (2004)). An aliquot of DMSO (350 μL) was added to the system as an aid to processing. The rig was sealed and pressurised with CO₂. The temperature and pressure were raised to approximately 40° C. and 2000 psi rendering the CO₂ a supercritical fluid. Whilst maintaining these conditions the PLGA and Degarelix were mixed for 30 min with a mechanical stirrer that formed part of the PGSS processing apparatus. Mixing was then ceased and the contents of the rig were subjected to 10 pressure cycles. Each pressure cycle lasted a total of 20 minutes and consisted of the pressure being decreased gradually to approximately 1000 psi and then immediately increased abruptly to re-achieve the desired system pressure. After completion of the 10 pressure cycles, the system was depressurised and the product was collected and ground to obtain a free flowing powder.

### Results

| Polymer | API | Processing Aid | VMD | d90 | d50 | d10 |
|---|---|---|---|---|---|---|
| PLGA | Degarelix 10 | DMSO | 68.67 | 125.05 | 58.67 | 22.58 |
| 8 kDa | wt % | | | | | |

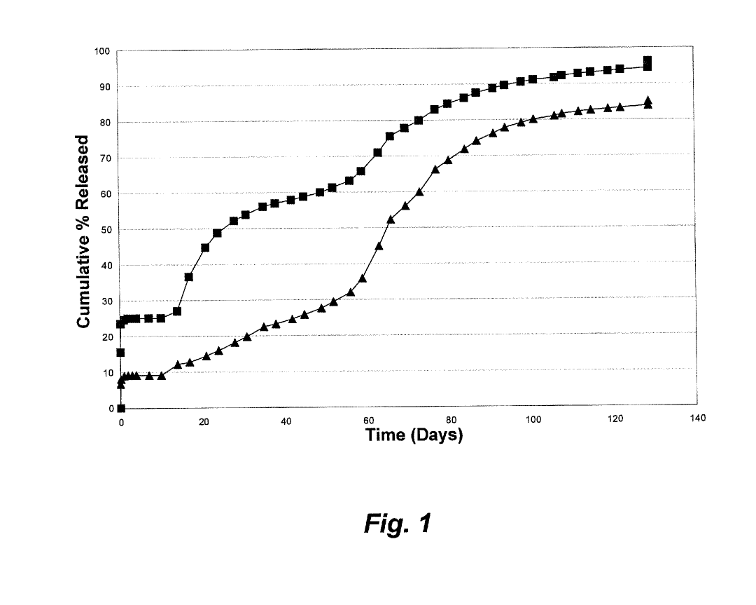

Release of degarelix from the polymer formulation was measured according to the method described above. The release profile observed is illustrated in FIG. 1.

### Reference Example 1b

### Degarelix Processed with DMSO but without Pressure Cycling

Although this example illustrates the principles of the process of the invention, it does not fall within the scope of the attached claims because degarelix is a GnRH antagonist.

### Method

The method used in this reference example was identical to that described in respect of Reference Example 1a above, except that:
(i) only 20 μL of DMSO (instead of 350 μL of DMSO) was added as a processing aid at the beginning of the process; and
(ii) the 10 pressure cycles were omitted.

### Results

Release of degarelix from the polymer formulation was measured according to the method described above. The release profile observed is illustrated in FIG. 1.

### Reference Example 2a

### Degarelix Processed without DMSO but with Pressure Cycling

Although this example illustrates the principles of the process of the invention, it does not fall within the scope of the attached claims because degarelix is a GnRH antagonist.

### Method

PLGA 75:25 (M_{w} 8 kDa, measured in THF relative to PS standards, 1.89 g) was mixed with Degarelix (0.21 g, 10 wt. %) by shaking/inverting the weighting vial containing both components. This mixture was loaded in to the supercritical fluid PGSS processing rig. The rig was sealed and pressurised with CO₂. The temperature and pressure were raised to approximately 40° C. and 2000 psi rendering the CO₂ a supercritical fluid. Whilst maintaining these conditions the PLGA/Degarelix were mixed for 30 min with a mechanical stirrer that formed part of the PGSS processing apparatus. Mixing was then ceased and the contents of the rig were subjected to 10 pressure cycles. Each pressure cycle lasted a total of 20 minutes and consisted of the pressure being decreased gradually to approximately 1000 psi and then immediately increased abruptly to re-achieve the desired system pressure. After completion of the 10 pressure cycles, the system was depressurised then the product was collected and ground to obtain a free flowing powder.

### Results

| Polymer | API | Processing Aid | VMD | d90 | d50 | d10 |
|---|---|---|---|---|---|---|
| PLGA | Degarelix | — | 68.58 | 113.57 | 66.41 | 27.23 |
| 8 kDa | 10 wt % | | | | | |

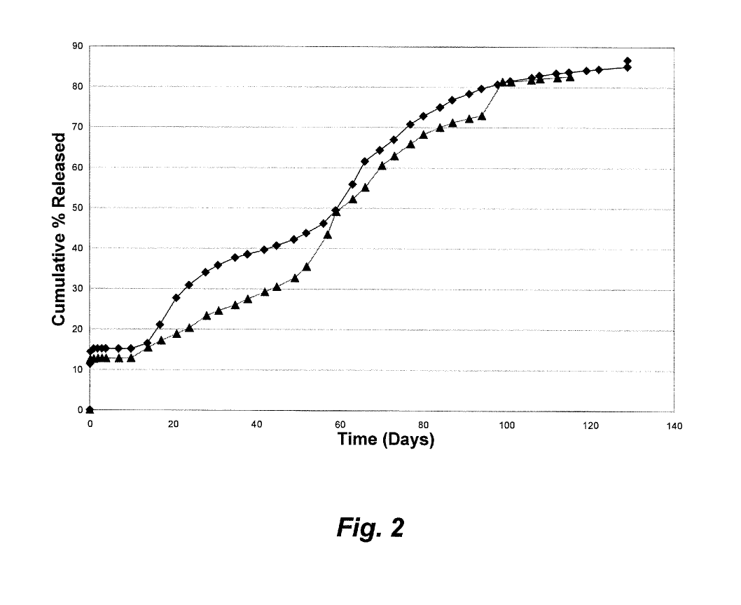

Release of degarelix from the polymer formulation was measured according to the method described above. The release profile observed is illustrated in FIG. 2.

### Reference Example 2b

### Degarelix Processed without DMSO or Pressure Cycling

Although this example illustrates the principles of the process of the invention, it does not fall within the scope of the attached claims because degarelix is a GnRH antagonist.

### Method

The method used in this reference example was identical to that described in respect of Reference Example 2a above, except that the 10 pressure cycles were omitted.

### Results

Release of degarelix from the polymer formulation was measured according to the method described above. The release profile observed is illustrated in FIG. 2.

### Example 3a

### Bovine Serum Albumin Processed with Pressure Cycling

### Method

A blend of 90% by weight of PLGA 50:50 and 10% by weight of PLA (M_{w} 11 and 9 kDa respectively, measured in THF relative to PS standards, 1.7 g) was mixed with Poloxamer 407 (0.1890 g, 0.9 w.t. %) and Bovine Serum Albumin (0.21 g, 10 w.t. %) by shaking/inverting the weighting vial containing all three components. This mixture was loaded in to the supercritical fluid PGSS processing rig. The system was sealed and pressurised with CO₂. The temperature and pressure were raised to approximately 40° C. and 2000 psi rendering the CO₂ a supercritical fluid. Whilst maintaining these conditions the PLGA/PLA/Poloxamer 407/BSA were mixed for 30 min with a mechanical stirrer that formed part of the PGSS processing apparatus. Mixing was then ceased and the contents of the rig were subjected to 10 pressure cycles. Each pressure cycle lasted a total of 20 minutes and consisted of the pressure being decreased gradually to approximately 1000 psi and then immediately increased abruptly to re-achieve the desired system pressure. After completion of the 10 pressure cycles, the mixture was atomised (by spraying through a nozzle, and collecting the powdered product in a cyclone, using 75 bar (7.5 MPa) back pressure) and collected yielding a course free flowing powder. The product was easily collected as a fine, free flowing white powder.

### Results

| Polymer | API | Processing Aid | VMD | d90 | d50 | d10 |
|---|---|---|---|---|---|---|
| PLGA/PLA | BSA | Poloxamer 407 | 89.56 | 173.91 | 74.51 | 26.63 |
| 9-11 kDa | 10 wt % | | | | | |

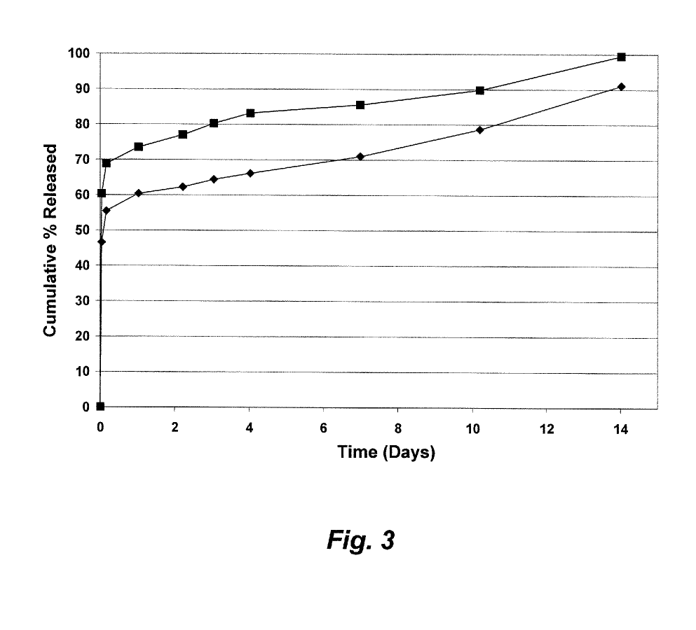

Release of BSA from the polymer formulation was measured according to the method described above. The release profile observed is illustrated in FIG. 3.

### Reference Example 3b

### Bovine Serum Albumin Processed without Pressure Cycling

### Method

The method used in this example was identical to that described in respect of Example 3a above, except that the 10 pressure cycles were omitted.

### Results

| Polymer | API | Processing Aid | VMD | d90 | d50 | d10 |
|---|---|---|---|---|---|---|
| PLGA/PLA | BSA | Poloxamer 407 | 110.94 | 219.36 | 92.13 | 29.84 |
| 9-11 kDa | 10 wt % | | | | | |

Release of BSA from the polymer formulation was measured according to the method described above. The release profile observed is illustrated in FIG. 3.

### ABBREVIATIONS

Arg Arginine
d10 Maximum diameter of at least 10% of particles in sample
d50 Maximum diameter of at least 50% of particles in sample
d90 Maximum diameter of at least 90% of particles in sample
DMSO Dimethylsulfoxide
Gly Glycine
Gly-NH₂ Glycinamide
His Histidine
Leu Leucine
Pro Proline
pyroGlu Pyroglutamic acid (5-oxoproline)
Ser Serine
Trp Tryptophan
Tyr Tyrosine
VMD Volume mean diameter

1. A process for preparing a solid polymer matrix containing a core material, said process comprising the steps of:
(a) providing a solid polymer, a core material and a fluid that is capable of existing in the supercritical state;
(b) in a mixing vessel, mixing the polymer, core material and fluid at
a temperature at or above T_{c} and
a pressure at or above P_{c},
such that the fluid is in the supercritical state, wherein T_{c} and P_{c} are the critical temperature and the critical pressure, respectively, for the fluid;
(c) without recovering the solid polymer matrix,
(i) converting the fluid in the vessel to a sub-critical state by reducing the pressure in said vessel to below P_{c} and/or reducing the temperature in said vessel to below T_{c}, and then
(ii) returning the fluid in the vessel to the supercritical state by increasing the pressure and/or the temperature in the vessel;
(d) optionally repeating step (c) one or more times; and
(e) releasing the pressure in the vessel and recovering solid polymer matrix containing the core material,
provided that the core material does not comprise any of gonadotropin releasing hormone (GnRH), a GnRH agonist and a GnRH antagonist.

2. A process according to claim 1, wherein
(a1) said providing comprises providing a solid polymer, a core material, a processing aid and a fluid that is capable of existing in the supercritical state;
(b1) said mixing comprises, in the mixing vessel, mixing the polymer, core material, processing aid and fluid at
a temperature at or above T_{c} and
a pressure at or above P_{c},
such that the fluid is in the supercritical state, wherein T_{c} and P_{c} are the critical temperature and the critical pressure, respectively, for the fluid.

3. A process according to claim 1, wherein the solid polymer comprises PLGA, PLA, or a combination of PLA and PLGA.

4. A process according to claim 1, wherein the inherent viscosity of the solid polymer is from about 0.05 to about 0.15 dL/g.

5. A process according to claim 1, wherein the solid polymer is a mixture of two or more of PLGA, PLA and a polyether.

6. A process according to claim 5, wherein the polyether is a block copolymer of ethylene glycol and propylene glycol and has the following formula, 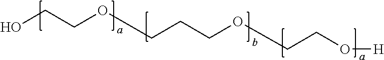
wherein each a is independently from 2 to 130 and b is from 15 to 67.

7. A process according to claim 5, wherein the polyether is present at from about 8 to about 15% of the combined weight of PLGA and PLA.

8. A process according to claim 1, wherein the solid polymer represents from about 45 to about 99.9% by weight of the combined weight of the solid polymer and the core material.

9. A process according to claim 1, wherein the core material has a solubility in the fluid used under the supercritical conditions selected for the process of less than 10 μg/mL.

10. A process according to claim 1, wherein the core material is selected from one or more of acarbose, acetyl cysteine, acetylcholine chloride, acitretin, acyclovir, alatrofloxacin, albendazole, albuterol, alendronate, amantadine hydrochloride, ambenomium, amifostine, amiloride hydrochloride, aminocaproic acid, amiodarone, amlodipine, amphetamine, amphotericin B, aprotinin, aripiprazole, atenolol, atorvastatin, atovaquone, atracurium besylate, atropine, axitinib, azithromycin, azithromycin, aztreonam, bacitracin, baclofen, becalermin, beclomethsone, belladona, benezepril, benzonatate, bepridil hydrochloride, betamethasone, bicalutanide, bleomycin sulfate, budesonide, bupropion, busulphan, butenafine, calcifediol, calciprotiene, calcitriol, camptothecan, candesartan, capecitabine, capreomycin sulfate, capsaicin, carbamezepine, carboplatin, carotenes, cefamandole nafate, cefazolin sodium, cefepime hydrochloride, cefixime, cefonicid sodium, cefoperazone, cefotetan disodium, cefotoxime, cefoxitin sodium, ceftizoxime, ceftriaxone, cefuroxime axetil, celecoxib, cephalexin, cephapirin sodium, cerivistatin, cetrizine, chlorpheniramine, cholecalciferol, cidofovir, cilostazol, cimetidine, cinnarizine, ciprofloxacin, ciprofloxacin, cisapride, cisplatin, cladribine, clarithromycin, clemastine, clidinium bromide, clindamycin and clindamycin derivatives, clomiphene, clomipramine, clondronate, clopidrogel, codeine, coenzyme Q10, colistimethate sodium, colistin sulfate, cromalyn sodium, cyclobenzaprine, cyclosporine, cytarabine, danaproid, danazol, dantrolene, deforoxamine, dexchlopheniramine, diatrizoate megluamine and diatrizoate sodium, diclofenac, dicoumarol, dicyclomine, didanosine, digoxin, dihydro epiandrosterone, dihydroergotamine, dihydrotachysterol, dirithromycin, dirithromycin, donepezil, dopamine hydrochloride, doxacurium chloride, doxorubicin, editronate disodium, efavirenz, elanaprilat, enoxacin, ephedrine, epinephrine, eposartan, ergocalciferol, ergotamine, erythromycin, esmol hydrochloride, essential fatty acid sources, etodolac, etoposide, famiciclovir, famotidine, fenofibrate, fentanyl, fexofenadine, finasteride, flucanazole, fludarabine, fluoxetine, flurbiprofen, fluvastatin, foscarnet sodium, fosphenytion, frovatriptan, furazolidone, gabapentin, ganciclovir, gemfibrozil, gentamycin, glibenclamide, glipizide, glyburide, glycopyrolate, glymepride, grepafloxacin, griseofulvin, halofantrine, ibuprofen, iloperidone, indinavir sulfate, ipratropium bromide, irbesartan, irinotecan, isofosfamide, isosorbide dinitrate, isotreinoin, itraconazole, ivermectin, japanese lamivudine, ketoconazole, ketorolac, L-thryroxine, lamotrigine, lanosprazole, lapatinib, leflunomide, leucovorin calcium, levofloxacin, lincomycin and lincomycin derivatives, lisinopril, lobucavir, lomefloxacin, loperamide, loracarbef, loratadine, lovastatin, lutein, lycopene, mannitol, medroxyprogesterone, mefepristone, mefloquine, megesterol acetate, mephenzolate bromide, mesalmine, metformin hydrochloride, methadone, methanamine, methotrexate, methoxsalen, methscopolamine, metronidazole, metronidazole, metroprolol, mezocillin sodium, miconazole, midazolam, miglitol, minoxidil, mitoxantrone, mivacurium chloride, montelukast, nabumetone, nalbuphine, naratiptan, nedocromil sodium, nelfinavir, neostigmine bromide, neostigmine methyl sulfate, neutontin, nifedipine, nilsolidipine, nilutanide, nitrofurantoin, nizatidine, norfloxacin, ofloxacin, olanzapine, olpadronate, omeprazole, oprevelkin, osteradiol, oxaprozin, oxytocin, paclitaxel, paliperidone, pamidronate disodium, pancuronium bromide, paricalcitol, paroxetine, paroxetine, pazopanib, pefloxacin, pentamindine isethionate, pentazocine, pentostatin, pentoxifylline, periciclovir, phentolamine mesylate, phenylalanine, physostigmine salicylate, pioglitazone, piperacillin sodium, pizofetin, polymixin B sulfate, pralidoxine chloride, pravastatin, prednisolone, pregabalin, probucol, progesterone, propenthaline bromide, propofenone, pseudo-ephedrine, pyridostigmine, pyridostigmine bromide, rabeprazole, raloxifene, refocoxib, repaglinide, residronate, ribavarin, rifabutine, rifapentine, rimantadine hydrochloride, rimexolone, risperidone, ritanovir, rizatriptan, rosigiltazone, salmetrol xinafoate, saquinavir, sertraline, sibutramine, sildenafil citrate, simvastatin, sirolimus, solatol, sorafenib, sparfloxacin, spectinomycin, spironolactone, stavudine, streptozocin, sumatriptan, sunitinib, suxamethonium chloride, tacrine, tacrine hydrochloride, tacrolimus, tamoxifen, tamsulosin, targretin, tazarotene, telmisartan, teniposide, terbinafine, terbutaline sulfate, terzosin, tetrahydrocannabinol, thiopeta, tiagabine, ticarcillin, ticlidopine, tiludronate, timolol, tirofibran, tizanidine, topiramate, topotecan, toremifene, tramadol, trandolapril, tretinoin, trimetrexate gluconate, troglitazone, trospectinomycin, trovafloxacin, trovafloxacin, tubocurarine chloride, ubidecarenone, urea, valaciclovir, valsartan, valsartan, vancomycin, vecoronium bromide, venlafaxine, vertoporfin, vigabatrin, vinblastin, vincristine, vinorelbine, vitamin A, vitamin B12, vitamin D, vitamin E, vitamin K, warfarin sodium, zafirlukast, zalcitabine, zanamavir, zidovudine, zileuton, zolandronate, zolmitriptan, zolpidem, zopiclone, and pharmaceutically acceptable salts thereof.

11. A process according to claim 1, wherein the core material is selected from one or more of:
insulin,
antihemophilic factor (Factor VIII),
Factor VII, Factor VIIa,
Factor IX,
growth hormones,
growth hormone releasing factor,
somatostatin,
glucagons,
parathyroid hormone,
calcitonin,
interleukins,
interleukin 1 receptor antagonist (IL-1Ra),
interferons (IFNs),
vascular endothelium growth factor (VEGF),
anti-VEGF antibodies or fragments thereof,
erythropoietins (EPOs),
heparin or low molecular weight heparin,
tissue plasminogen activator (t-PA),
platelet derived growth factors (PDGFs),
cyclosporin A and cyclosporin A analogs,
oxytocin,
enkephalin,
tyrotropin releasing hormone,
vasopressin and vasopressin analogs,
catalase,
superoxide dismutase,
glatiramer acetate,
bone morphogenetic protein (BMP),
colony stimulating factors (CSFs),
tumor necrosis factors,
TNFα inhibitors,
melanocyte stimulating hormone (MSH),
glucagon-like peptide-1 (GLP-1),
glucagon-like peptide-2 (GLP-2),
katacalcin,
cholecystekinin-12,
cholecystekinin-8,
exendin,
gonadoliberin-related peptide,
insulin-like protein,
leucine-enkephalin,
methionine-enkephalin,
leumorphin,
neurophysin,
copeptin,
neuropeptide Y,
neuropeptide AF,
PACAP-related peptide,
pancreatic hormone,
peptide YY,
urotensin,
intestinal peptide,
adrenocorticotropic peptide,
epidermal growth factor,
prolactin,
gastrin,
tetragastrin,
pentagastrin,
endorphins,
angiotensins,
thyrotropin releasing hormone,
heparinase,
alglucerase,
asparaginase,
cortocotropin,
denileukin diftitox,
dornase alpha,
streptokinase,
urokinase,
cosyntropin,
desmopressin,
octreotide acetate,
pramlintide,
sincalide,
enzymes,
glycoproteins, and
antigens derived from or consisting of live or inactivated microorganisms.

12. A process according to claim 1, wherein the core material is a recombinant hGH, or an analogue thereof.

13. A process according to claim 1, wherein the core material is selected from the list consisting of risperidone; paliperidone; aripiprazole; iloperidone; olanzapine; interferon alpha; interferon beta; glatiramer acetate; erythropoietin; anti-VEGF antibodies or fragments thereof; anti-TNFα antibodies or fragments thereof; Factor VII; Factor VIIa; Factor IX; BMP; GLP-1, and analogues of those materials.

14. A process according to claim 1, wherein the core material represents from about 5% to about 15% of the combined weight of the solid polymer and the core material.

15. A process according to claim 1, wherein the fluid is carbon dioxide.

16. A process according to claim 1, wherein the supercritical conditions achieved during process step (b) are maintained for a time period of from about 10 to about 60 minutes.

17. A process according to claim 1, wherein during step (b) the contents of the mixing vessel are stirred whilst the fluid is in the supercritical state.

18. A process according to claim 1, wherein step (c) comprises the steps of:
(ia) converting the fluid in the vessel to a sub-critical state by reducing the pressure in said vessel to below P_{c}, and then
(iia) returning the fluid in the vessel to the supercritical state by increasing the pressure in the vessel.

19. A process according to claim 1, wherein in step (c) the pressure is reduced to a minimum within the range of from about 5.1 MPa to 97% of P_{c} for the fluid used in the process.

20. A process according to claim 1, wherein when the fluid is carbon dioxide and the pressure in step (c) is reduced to minimum within the range of about 6.5 to about 7.0 MPa.

21. A process according to claim 1, wherein step (c) is effected in the absence of active mixing of the contents of the mixing vessel.

22. A process according to claim 1, wherein the period of time to complete each repetition of steps (i) and (ii) together of step (c) is from about 10 to about 30 minutes.

23. A process according to claim 1, wherein step (d) comprises from 4 to 10 repetitions of the cycle of step (c).

24. A process according to claim 1, wherein in step (e) the contents of mixing vessel are discharged through a nozzle or like orifice into a second vessel at lower pressure.

25. A solid polymer matrix containing a core material that is obtainable by a process according to claim 1, provided that the core material does not comprise any of gonadotropin releasing hormone (GnRH), a GnRH agonist and a GnRH antagonist.

26. A process for preparing a pharmaceutical composition comprising a solid polymer matrix that contains a core material, wherein the core material is a biologically active material, provided that the core material does not comprise any of gonadotropin releasing hormone (GnRH), a GnRH agonist and a GnRH antagonist,
said process comprising a process according to claim 1, followed by a step of formulating the solid polymer matrix for pharmaceutical use.
